SEQUENCE LISTING (12 sequences)

SEQ ID NO: 1 - protein, 13 aa, Artificial Sequence
  CPP sequence of HIV-Tat protein
PGRKKRRQRRPPQ

SEQ ID NO: 2 - protein, 16 aa, Artificial Sequence
  CPP sequence of penetratin
RQIKIWFQNRRMKWKK

SEQ ID NO: 3 - protein, 27 aa, Artificial Sequence
  CPP sequence of transportan
GWTLNSAGYLLGKINLKALAALAKKIL

SEQ ID NO: 4 - protein, 34 aa, Artificial Sequence
  CPP sequence of HSV-1 structural protein
DAATATRGRSAASRPTERPRAPARSASRPRRPVD

SEQ ID NO: 5 - protein, 27 aa, Artificial Sequence
  CPP sequence of HIV Gp41-SV40 NLS
GALFLGFLGAAGSTMGAWSQPKKKRKV

SEQ ID NO: 6 - protein, 21 aa, Artificial Sequence
  CPP sequence SV40-NLS
KETWWETWWTEWSQPKKKRKV

SEQ ID NO: 7 - protein, 12 aa, Artificial Sequence
  CPP sequence
KALAKALAKALA

SEQ ID NO: 8 - protein, 18 aa, Artificial Sequence
  CPP sequence
VRLPPPVRLPPPVRLPPP

SEQ ID NO: 9 - protein, 20 aa, Artificial Sequence
  CPP sequence
GLFRALLRLLRSLWRLLLRA

SEQ ID NO: 10 - protein, 22 aa, Artificial Sequence
  CPP sequence from human calcitonin
LGTYTQDFNKTFPQTAIGVGAP

SEQ ID NO: 11 - protein, 18 aa, Artificial Sequence
  CPP sequence from protegrin
RGGRLSYSRRRFSTSTGR

SEQ ID NO: 12 - protein, 18 aa, Artificial Sequence
  CPP sequence from murine VE-cadherin
LLIILRRRIRKQAHAHSK

(19) United States
(12) Patent Application Publication
(10) Pub. No.: US 2017/0233745 A1
(43) Pub. Date: Aug. 17, 2017

(54) UREIDOPYRIMIDONE SUPRAMOLECULAR COMPLEXES FOR COMPOUND DELIVERY INTO CELLS

(71) Applicant: TU Eindhoven, Eindhoven (NL)

(72) Inventors: Patricia Yvonne Wilhelmina DANKERS, Eindhoven (NL); Lorenzo ALBERTAZZI, Eindhoven (NL); Maarten Herman BAKKER, Eindhoven (NL)

(21) Appl. No.: 15/504,875

(22) PCT Filed: Aug. 19, 2015
(86) PCT No.: PCT/NL2015/050583

§ 371 Date: Feb. 17, 2017

(30) Foreign Application Priority Data
Aug. 20, 2014 (EP) EP 14181673.6

(51) Int. Cl. C12N 15/113; C07D 239/47; A61K 9/16; A61K 49/00

(52) U.S. Cl. CPC C12N 15/1138; A61K 49/0073; C07D 239/47; A61K 9/16; C12N 2310/14; C12N 2310/351; C12N 2320/32

(57) ABSTRACT

The present invention is directed to particle comprising a supramolecular complex comprising a monofunctional and/or a bifunctional subunit comprising a quadruple hydrogen bonding unit, an apolar linker, an urea group, and a polyethyleneglycol linker. The monofunctional subunits comprise a functional group. The particles are very suitable as drug delivery system as they bind and enter the cell and may have slow release properties.

The invention relates to supramolecular complexes that are able to deliver compounds into the cells. The invention relates also to hydrogels with the same properties but also with additional properties. The invention furthermore relates to cell delivery systems and labeling agents.

### BACKGROUND OF THE INVENTION

Gene therapy has been successfully used for disease such as retinal disease Leber's congenital amaurosis, X-linked SCID, ADA-SCID, adrenoleukodystrophy, chronic lymphocytic leukemia (CLL), acute lymphocytic leukemia (ALL), multiple myeloma, haemophilia, and Parkinson's disease. In 2012, Glybera became the first viral gene-therapy treatment to be approved in Europe. The treatment uses an adeno-associated virus to deliver a working copy of the LPL (lipoprotein lipase) gene to muscle cells. Gene therapy has also high potential for severe diseases caused by single-gene defects, such as cystic fibrosis, haemophilia, muscular dystrophy, thalassemia, and sickle cell anemia.

In gene therapy, DNA or RNA must be administered to the patient, get to the cells that need repair, and enter the cell to have its effect. As DNA and RNA cell internalization is not very effective a carrier is often needed. Successful therapies have mostly been dependent on viral vectors. Although viral vectors may be effective in delivery of nucleic acids into a cell, they have several drawbacks, such as that they are difficult to make, difficult to handle, costly and there is a risk of erroneous integration, which may cause cancer. There is thus still a need for effective carriers able to deliver nucleic acids, and especially RNA, such as antisense RNA, into the right cell population. Preferably such effective carriers are also cost effective, easy to handle and/or stable upon storage.

### SUMMARY OF THE INVENTION

In a first aspect, the invention is directed to a particle comprising a supramolecular complex comprising a monofunctional subunit with formula (I)

4H-L₁-F₁-L₂-F₂—P-E-Z (I)

and/or a bifunctional subunit with formula (II):

4H-L₁-F₁-L₂-F₂-G-F₂-L₂-F₁-L₁-4H (II)

wherein
4H is a quadruple hydrogen bonding unit
L₁ and L₂ is selected from the group comprising C₁₋₅₀ alkyl, or C₂₋₅₀ alkenyl;

### F₁ is —NH—C(═O)—NH;

F₂ is selected from the group consisting of —NRₐ—C(X)—NRₐ— or —NRₐ—C(X)—X—,

### X is O or S;

Rₐ is hydrogen, or C₁₋₁₂ alkyl;
G is a polyethyleneglycol linker with a molecular weight of at least 500 Dalton.
P is a polyethyleneglycol linker with 0 to 1000 ethyleneglycol monomers
E is a direct bond, linker L_{E}, linker P_{E}, or combinations of L_{E} and P_{E} linkers;
L_{E} is a linker as defined with L₁ or L₂;
P_{E} is a polyethyleneglycol linker as defined with polyethyleneglycol linker P;
Z is a functional group selected from the group comprising a neutral moiety, ionic moiety, peptide, therapeutic moiety, imaging agent, fluorescent moiety, targeting moiety, endosomal escape agent moiety, cell-penetrating peptides, antigen, adjuvant, antibody.

In a preferred embodiment of the present invention and/or embodiments thereof, at least 10 subunits of formula (I) are present in the particle.

In a preferred embodiment of the present invention and/or embodiments thereof, at least 10% of the subunits are cationic.

In a preferred embodiment of the present invention and/or embodiments thereof, the z potential of the particle is between 0 and +50 v.

In a preferred embodiment of the present invention and/or embodiments thereof, the hydrodynamic diameter of the particle is between 0.2 and 1000 nm.

In a preferred embodiment of the present invention and/or embodiments thereof, the monofunctional subunit has formula (III)

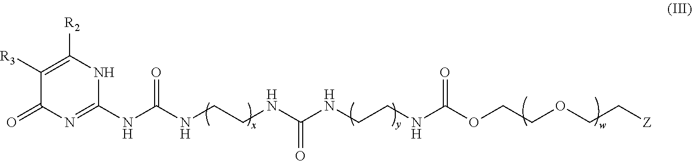

x is an integer from 1 to 50,
y is an integer from 1 to 50
w is an integer from 0 to 1000,
R₂, R₃ is each independently a hydrogen, C₁₋₂₄alkyl, C₂₋₂₄alkenyl, C₂₋₂₄alkynyl, C₃₋₁₂-cycloalkyl.

In a preferred embodiment of the present invention and/or embodiments thereof, the particle comprises at least one monofunctional subunit with formula (I)

4H-L₁-F₁-L₂-F₂—P-E-Z (I)

And at least one bifunctional subunit with formula (II):

4H-L₁-F₁-L₂-F₂-G-F₂-L₂-F₁-L₁-4H (II)

In a preferred embodiment of the present invention and/or embodiments thereof, the bifunctional subunit is present in an amount of at least 2 wt %.

In a preferred embodiment of the present invention and/or embodiments thereof the particle is in the form of a hydrogel.

In a second aspect, the invention is directed to a process for making a particle according to aspects of the invention and/or embodiments thereof comprising the step
i) adding a subunit as defined in any of the aspects and/or embodiments to water.

In a third aspect, the invention is directed to a method for entering or labelling a cell using a particle according to aspects of the invention and/or embodiments thereof.

In a fourth aspect, the invention is directed to use of a particle according to aspects of the invention and/or embodiments thereof as drug delivery system.

In a fifth aspect, the invention is directed to use of a particle according to aspects of the invention and/or embodiments thereof as imaging agent.

In a sixth aspect, the invention is directed to use of a particle according to aspects of the invention and/or embodiments thereof in the form of hydrogel for prolonged release system.

In a seventh aspect, the invention is directed to use of a particle according to aspects of the invention and/or embodiments thereof in the form of hydrogel as mechanical support for damaged tissue.

### DETAILED DESCRIPTION

### Figure Legend

FIG. 1. Proposed assembly mechanism of various subunits into supramolecular stacks upon injection into water. Different subunits are used such as neutral subunits (blue rod), cationic subunits (yellow dot), and subunits with a dye (red dots).

FIG. 2. Nile Red (NR) fluorescent intensity upon encapsulation by particles consisting of purely neutral subunits, 20% cationic subunits+80% neutral subunits, 50% cationic subunits+50% neutral subunits, 80% cationic subunits+20% neutral subunits or 100% cationic subunits.

FIG. 3A: Autocorrelation functions from dynamic light scattering (DLS) measurements at an angle of 102 degrees for neutral, 20% cationic, 50% cationic, 80% cationic and full cationic particles. Non-connecting markers represent the autocorrelation data and the solid line the fitted stretched exponential. B: To obtain a value for the dispersity of the sample a stretched exponential was fitted.

FIG. 4. Neutral, 50% cationic and full cationic particles were assembled in water and their z-potential was measured at a pH of approximately 7.0.

FIG. 5. Occurrence of FRET effect upon encapsulation of NR and co-assembly of the Cy5 reporter monomer for neutral (A), 50% cationic (B) and full cationic (C) stacks.

FIG. 6. Interaction of neutral, 50% cationic and full cationic particle with HK2 cells.

FIG. 7. Four images acquired at different time points during particle internalization.

FIG. 8. Particle internalization after 4 hours incubation, showing internalization for 50% and full cationic stacks but not for the neutral. Staining of live and dead cells using respectively Calcein-AM (green) and Ethidium bromide homodimer-1 (red) shows no cytotoxicity up to this point.

FIG. 9. MTT assay conducted to quantify HK2 cell viability after 24 hours of particle incubation at various concentrations. Values are mean±SD, n=7.

FIG. 10. Neutral, 50% cationic and full cationic particle in gel electrophoresis. Left side of the gel represents the conventional preparation method while the right side represents the templating method. N/P ratios of 0 (negative control) to 20 were evaluated.

FIG. 11. Autocorrelation functions from DLS measurements at an angle of 102 degrees for 50% and full cationic particles prepared via two preparation methods. Non-connecting markers represent the autocorrelation data and the solid line is the fitted stretched exponential.

FIG. 12. Internalization of siRNA. Images acquired after 1 hour of incubation with particle-siRNA complexes without washing the samples.

FIG. 13. Internalization of siRNA Images after washing of the samples with PBS at 2 hours of incubation with particle assemblies-siRNA complexes.

FIG. 14: Results of silencing the TGFBR1 gene. 50% Cationic particle, full cationic particles were prepared via the conventional preparation method at an N/P ratio of 10. Samples are normalized versus untreated cells and represent mean±SD, n=3.

### DEFINITIONS

A supramolecular complex is a complex made of assembled molecular subunits or components. The forces responsible for the spatial organization may vary from weak (intermolecular forces, electrostatic or hydrogen bonding) to strong (covalent bonding), provided that the degree of electronic coupling between the molecular component remains small with respect to relevant energy parameters of the component. A supramolecular complex is different from a chemical complex in that in a supramolecular complex the interactions between subunits are mainly the weaker and reversible non-covalent interactions between molecules, whereas in traditional chemistry the interactions are covalent. These interactions in supramolecular complexes include hydrogen bonding, metal coordination, hydrophobic forces, van der Waals forces, pi-pi interactions and electrostatic effects. Important concepts that are indicative of supramolecular chemistry include molecular self-assembly, folding, molecular recognition, host-guest chemistry, mechanically-interlocked molecular architectures, and dynamic covalent chemistry. For the purpose of the present invention, the subunits form a supramolecular complex by self-assembly and the forces holding the subunits together are preferably hydrogen bonding.

According to the present invention, a particle is a small localized object to which can be ascribed several physical and/or chemical properties such as volume, mass, charge etc. Particles may be of micro-size or nano-size. Particles may be essentially spherical, or may have an elongated form. The particles of the present invention comprise a supramolecular structure of subunits as defined. The monomeric subunits (I) may self-assemble into dimers, and the dimers may form aggregates and thus form the supramolecular structure which make the particle. The bifunctional subunits (II) may also aggregate and form supramolecular structures, thus forming the particle. Also combinations of the monomeric subunits (I) and the bifunctional subunits (II) may aggregate and form supramolecular structures, thus forming the particle. The particle may thus be an aggregate and/or a supramolecular structure. Suitably the particle is an aggregate of monomeric subunits (I), an aggregate of dimeric subunits, an aggregate of bifunctional subunits (II) or an aggregate of any combination of two or more subunits selected from the group consisting of monomeric subunits (I), dimeric subunits, and bifunctional subunits (II). Optionally the particle is a supramolecular structure of monomeric subunits (I), a supramolecular structure of dimeric subunits, a supramolecular structure of bifunctional subunits (II) or a supramolecular structure of any combination of two or more subunits selected from the group consisting of monomeric subunits (I), dimeric subunits, and bifunctional subunits (II). The particle, aggregate, or supramolecular structure of the present invention and/or embodiments thereof may encapsulate compounds and/or the subunits carry functional groups. The functional groups may be covalently bound to the subunit or via other forces such as hydrogen bonding, electrostatic forces, van der waal forces, pi-pi interactions, or hydrophobic forces.

The particles, aggregate, or supramolecular structure of the present invention may form stacks and may have an elongated form or may form spheres. The particles, aggregate, or supramolecular structure of the present invention may form fibers. Suitably the particles, aggregates, or supramolecular structures of the present invention and/or embodiments thereof are of nanosize.

For the purpose of the present invention, percentage, %, may be molar percentage, mol % or weight percentage, wt %. For the purpose of the present invention the amount of monomer in the particle is indicated in mol % unless otherwise indicated. For the purpose of the present invention the amount of material in gels the amount is indicated as wt % unless otherwise indicated.

For the purpose of the present invention an alkyl is a saturated aliphatic group comprising of carbon atoms and may be branched, cyclic or linear. The alkyl may comprise heteroatoms such as O, N and S, preferably O and N, preferably O, preferably N. An alkyl with a heteroatom is referred to as heteroalkyl. The alkyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. R₅ maybe H, C₁₋₆alkyl, C₂₋₆alkenyl, C₂₋₆alkynyl, C₅₋₁₂ aryl. In preferred embodiments of the invention and/or embodiments thereof, alkyl is C₁₋₅₀ alkyl, C₁₋₄₀ alkyl, C₁₋₃₅ alkyl, C₁₋₃₀ alkyl, C₁₋₂₄ alkyl, C₁₋₂₀ alkyl, C₁₋₁₈ alkyl, C₁₋₁₆ alkyl, C₁₋₁₄ alkyl, C₁₋₁₂ alkyl, C₁₋₁₀ alkyl, C₁₋₉ alkyl, C₁₋₈ s alkyl, C₁₋₇ alkyl, C₁₋₆ alkyl, C₁₋₅ alkyl, C₁₋₄ alkyl, C₁₋₃ alkyl, or C₁₋₂ alkyl.

For the purpose of the present invention an alkenyl is an aliphatic group comprising of carbon atoms comprising one or more unsaturated double bonds and may be branched, cyclic or linear. The alkenyl may comprise heteroatoms such as O, N and S, preferably O and N, preferably O, preferably N. An alkenyl with a heteroatom is referred to as heteroalkenyl. The alkenyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. R₅ maybe H, C₁₋₆alkyl, C₂₋₆alkenyl, C₂₋₆alkynyl, C₅₋₁₂ aryl. In preferred embodiments of the invention and/or embodiments thereof, alkenyl is C₂₋₅₀ alkenyl, C₂₋₄₀ alkenyl, C₂₋₃₅ alkenyl, C₂₋₃₀ alkenyl, C₂₋₂₄ alkenyl, C₂₋₂₀ alkenyl, C₂₋₁₈ alkenyl, C₂₋₁₆ alkenyl, C₂₋₁₄ alkenyl, C₂₋₁₂ alkenyl, C₂₋₁₀ alkenyl, C₂₋₉ alkenyl, C₂₋₈ alkenyl, C₂₋₇ alkenyl, C₂₋₆ alkenyl, C₂₋₅ alkenyl, C₂₋₄ alkenyl, C₂₋₃ alkenyl, or C₂-alkenyl.

For the purpose of the present invention an alkynyl is an aliphatic group comprising of carbon atoms comprising one or more unsaturated triple bonds and may be branched, cyclic or linear. The alkynyl may comprise heteroatoms such as O, N and S, preferably O and N, preferably O, preferably N. An alkynyl with a heteroatom is referred to as heteroalkynyl. The alkynyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. R₅ maybe H, C₁₋₆alkyl, C₂₋₆alkenyl, C₂₋₆alkynyl, C₅₋₁₂ aryl. In preferred embodiments of the invention and/or embodiments thereof, alkynyl is C₂₋₅₀ alkynyl, C₂₋₄₀ alkynyl, C₂₋₃₅ alkynyl, C₂₋₃₀ alkynyl, C₂₋₂₄ alkynyl, C₂₋₂₀ alkynyl, C₂₋₁₈ alkynyl, C₂₋₁₆ alkynyl, C₂₋₁₄ alkynyl, C₂₋₁₂ alkynyl, C₂₋₁₀ alkynyl, C₂₋₉ alkynyl, C₂₋₈ alkynyl, C₂₋₇ alkynyl, C₂₋₆ alkynyl, C₂₋₅ alkynyl, C₂₋₄ alkynyl, C₂₋₃ alkynyl, or C₂-alkynyl.

For the purpose of the present invention an aryl refers to any functional group or substituent derived from an aromatic ring, and may comprise heteroatoms such as O, N and S, preferably O and N, preferably O, preferably N. Aryls with heteroatoms are also referred to as heteroaryl. The aryl may contain 5 to 12 atoms. The aryl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. R₅ maybe H, C₁₋₆alkyl, C₂₋₆alkenyl, C₂₋₆alkynyl, C₅₋₁₂ aryl. Aryl may be any of the group consisting of phenyl, naphthyl, thienyl, indolyl, tolyl, xylyl, furyl, and pyridyl. Preferred aryls are phenyl, tolyl and pyridyl.

Alkoxy or alkyloxy means an alkyl-O— group in which the alkyl group is as previously described. In preferred embodiments of the invention and/or embodiments thereof, alkoxy is C₂₋₅₀ alkoxy, C₂₋₄₀ alkoxy, C₂₋₃₅ alkoxy, C₂₋₃₀ alkoxy, C₂₋₂₄ alkoxy, C₂₋₂₀ alkoxy, C₂₋₁₈ alkoxy, C₂₋₁₆ alkoxy, C₂₋₁₄ alkoxy, C₂₋₁₂ alkoxy, C₂₋₁₀ alkoxy, C₂₋₉ alkoxy, C₂₋₈ alkoxy, C₂₋₇ alkoxy, C₂₋₆ alkoxy, C₂₋₅ alkoxy, C₂₋₄ alkoxy, C₂₋₃ alkoxy, or C₂-alkoxy. The alkoxy may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. Exemplary alkoxy groups include methoxy, ethoxy, n-propoxy, propoxy, n-butoxy and heptoxy.

Alkylthio means an alkyl-S— group in which the alkyl group is as previously described. The alkylthio may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. In preferred embodiments of the invention and/or embodiments thereof, alkthio is C₂₋₅₀ alkthio, C₂₋₄₀ alkthio, C₂₋₃₅ alkthio, C₂₋₃₀ alkthio, C₂₋₂₄ alkthio, C₂₋₂₀ alkthio, C₂₋₁₈ alkthio, C₂₋₁₆ alkthio, C₂₋₁₄ alkthio, C₂₋₁₂ alkthio, C₂₋₁₀ alkthio, C₂₋₉ alkthio, C₂₋₈ alkthio, C₂₋₇ alkthio, C₂₋₆ alkthio, C₂₋₅ alkthio, C₂₋₄ alkthio, C₂₋₃ alkthio, or C₂-alkthio.

Exemplary alkylthio groups include methylthio, ethylthio, propylthio and heptylthio.

Oxyalkylenyloxy means a —O— alkyl-O— group in which the alkyl group is as previously described. An exemplary alkylenedioxy group is —O—CH₂—O—. The oxyalkylenyloxy may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

Alkoxycarbonyl means an alkyl-O—CO— group in which the alkyl group is as previously described. The alkoxycarbonyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

In preferred embodiments of the invention and/or embodiments thereof, alkoxycarbonyl is C₂₋₅₀ alkoxycarbonyl, C₂₋₄₀ alkoxycarbonyl, C₂₋₃₅ alkoxycarbonyl, C₂₋₃₀ alkoxycarbonyl, C₂₋₂₄ alkoxycarbonyl, C₂₋₂₀ alkoxycarbonyl, C₂₋₁₈ alkoxycarbonyl, C₂₋₁₆ alkoxycarbonyl, C₂₋₁₄ alkoxycarbonyl, C₂₋₁₂ alkoxycarbonyl, C₂₋₁₀ alkoxycarbonyl, C₂₋₉ alkoxycarbonyl, C₂₋₈ alkoxycarbonyl, C₂₋₇ alkoxycarbonyl, C₂₋₆ alkoxycarbonyl, C₂₋₅ alkoxycarbonyl, C₂₋₄ alkoxycarbonyl, C₂₋₃ alkoxycarbonyl, or C₂-alkoxycarbonyl.

Exemplary alkoxycarbonyl groups include methoxycarbonyl and ethoxycarbonyl.

Acyl means an H—CO— or alkyl-CO— group in which the alkyl group is as previously described. Acyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

Preferred acyls contain a lower alkyl. In preferred embodiments of the invention and/or embodiments thereof, acyl is C₂₋₅₀ acyl, C₂₋₄₀ acyl, C₂₋₃₅ acyl, C₂₋₃₀ acyl, C₂₋₂₄ acyl, C₂₋₂₀ acyl, C₂₋₁₈ acyl, C₂₋₁₆ acyl, C₂₋₁₄ acyl, C₂₋₁₂ acyl, C₂₋₁₀ acyl, C₂₋₉ acyl, C₂₋₈ acyl, C₂₋₇ acyl, C₂₋₆ acyl, C₂₋₅ acyl, C₂₋₄ acyl, C₂₋₃ acyl, or C₂-acyl.

Exemplary acyl groups include formyl, acetyl, propanoyl, 2-methyipropanoyl, butanoyl and palmitoyl.

Acylamino is an acyl-NH— group wherein acyl is as defined herein. Acyl amino may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

In preferred embodiments of the invention and/or embodiments thereof, acylamino is C₂₋₅₀ acylamino, C₂₋₄₀ acylamino, C₂₋₃₅ acylamino, C₂₋₃₀ acylamino, C₂₋₂₄ acylamino, C₂₋₂₀ acylamino, C₂₋₁₈ acylamino, C₂₋₁₆ acylamino, C₂₋₁₄ acylamino, C₂₋₁₂ acylamino, C₂₋₁₀ acylamino, C₂₋₉ acylamino, C₂₋₈ acylamino, C₂₋₇ acylamino, C₂₋₆ acylamino, C₂₋₅ acylamino, C₂₋₄ acylamino, C₂₋₃ acylamino, or C₂-acylamino.

Alkylsulfonyl means an alkyl-SO— group in which the alkyl group is as previously described. Alkylsulfonyl may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

In preferred embodiments of the invention and/or embodiments thereof, alkylsulfonyl is C₂₋₅₀ alkylsulfonyl, C₂₋₄₀ alkylsulfonyl, C₂₋₃₅ alkylsulfonyl, C₂₋₃₀ alkylsulfonyl, C₂₋₂₄ alkylsulfonyl, C₂₋₂₀ alkylsulfonyl, C₂₋₁₈ alkylsulfonyl, C₂₋₁₆ alkylsulfonyl, C₂₋₁₄ alkylsulfonyl, C₂₋₁₂ alkylsulfonyl, C₂₋₁₀ alkylsulfonyl, C₂₋₉ alkylsulfonyl, C₂₋₈ alkylsulfonyl, C₂₋₇ alkylsulfonyl, C₂₋₆ alkylsulfonyl, C₂₋₅ alkylsulfonyl, C₂₋₄ alkylsulfonyl, or C₂₋₃ alkylsulfonyl.

Preferred groups are those in which the alkyl group is lower alkyl, such as C₂₋₁₂alkylsulfonyl, or such as C₂₋₆alkylsulfonyl, or such as C₂₋₄alkylsulfonyl.

For the purpose of the present invention an alkyl ether is a alkyl group as defined above comprising one or more —O— group and may be branched, cyclic or linear. The akyl ether may be substituted with groups selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl. R₅ maybe H, C₁₋₆alkyl, C₂₋₆alkenyl, C₂₋₆alkynyl, C₅₋₁₂ aryl. In preferred embodiments of the invention and/or embodiments thereof, alkyl ether is C₂₋₅₀ alkyl ether, C₂₋₄₀ alkyl ether, C₂₋₃₅ alkyl ether, C₂₋₃₀ alkyl ether, C₂₋₂₄ alkyl ether, C₂₋₂₀ alkyl ether, C₂₋₁₈ alkyl ether, C₂₋₁₆ alkyl ether, C₂₋₁₄ alkyl ether, C₂₋₁₂ alkyl ether, C₂₋₁₀ alkyl ether, C₂₋₉ alkyl ether, C₂₋₈ alkyl ether, C₂₋₇ alkyl ether, C₂₋₆ alkyl ether, C₂₋₅ alkyl ether, C₂₋₄ alkyl ether, or C₂₋₃ alkyl ether.

For the purpose of the present invention a halo group is a halogen group and may comprise iodine, chlorine, bromine, or fluorine. Preferably the halo is a chlorine or a bromine, preferably a chlorine, preferably a bromine, preferably a fluorine.

Lower alkyl means C₁-C₁₀ and may be straight or branched alkyl as well as C₃-C₈ cycloalkyl. Preferably C₁-C₆, more preferably C₁-C₄.

The term “antimicrobial activity” is defined herein as an activity which is capable of killing or inhibiting growth of microbial cells. In the context of the present invention the term “antimicrobial” is intended to mean that there is a bactericidal and/or a bacteriostatic and/or fungicidal and/or fungistatic effect and/or a virucidal effect, wherein the term “bactericidal” is to be understood as capable of killing bacterial cells. The term “fungicidal” is to be understood as capable of killing fungal cells. The term “fungistatic” is to be understood as capable of inhibiting fungal growth, i.e. inhibiting growing fungal cells. The term “virucidal” is to be understood as capable of inactivating virus. The term “microbial cells” denotes bacterial or fungal cells (including yeasts).

The present invention is directed to a particle comprising a supramolecular complex comprising a monofunctional subunit with general formula (I):

4H-L₁-F₁-L₂-F₂—P-E-Z (I)

and/or a bifunctional subunit with formula (II):

4H-L₁-F₁-L₂-F₂-G-F₂-L₂-F₁-L₁-4H (II)

The present invention may be directed to a particle comprising a supramolecular complex comprising a monofunctional subunit with general formula (I):

4H-L₁-F₁-L₂-F₂—P-E-Z (I).

The present invention may be directed to a particle comprising a supramolecular complex comprising a bifunctional subunit with formula (II):

4H-L₁-F₁-L₂-F₂-G-F₂-L₂-F₁-L₁-4H (II)

The present invention may be directed to a particle comprising a supramolecular complex comprising a monofunctional subunit with general formula (I):

4H-L₁-F₁-L₂-F₂—P-E-Z (I)

and a bifunctional subunit with formula (II):

4H-L₁-F₁-L₂-F₂-G-F₂-L₂-F₁-L₁-4H (II).

The subunit of the invention comprises a 4H unit which denotes a quadruple hydrogen bonding unit that is capable of forming at least four H-bridges with each other. The hydrogen bonding leads to physical interactions between two subunits. The physical interactions originate from multiple hydrogen bonding interactions (supramolecular interactions) between the self-complementary 4H units comprising at least four hydrogen bonds in a row. Units capable of forming at least four hydrogen bonds, i. e. quadruple hydrogen bonding units, are in this patent application abbreviated as 4H-units, 4H-elements or structural elements (4H) and are used in this patent application as interchangeable terms. Sijbesma et al. (U.S. Pat. No. 6,320,018; Science, 278, 1601; incorporated by reference herein) discloses such self-complementary units which are based on 2-ureido-4-pyrimidones (UPy). Such UPy units have been used to create supramolecular polymers. Supramolecular polymers consist of subunits that are held together by reversible and highly directional secondary interactions—that is, non-covalent bonds, such as hydrogen bonds (de Greef & Meijer Nature 453, 171-173, 2008). This is different from conventional polymers wherein the interaction between subunits is mainly covalent. A subunit with at least two 4H groups such as a UPy group is under the proper conditions able to self-assemble into polymers. Also conventional polymers have been derivatised with 4H groups such as UPy side chains to add properties to the polymers (Feldman et al, macromolecules 2009, 42, 9072-9081). UPy-modified polyethyleneglycol (PEG) subunits have been shown to be able to form a hydrogel (Dankers et al. Adv. Mater. 2012, 24, 2703-2703) and have been used as hydrogel carrier for guided, local catheter injection in infarcted myocardium (Bastings et al. Adv. Healthcare Mater., 2014, 3, 70-78).

In general, the 4H unit that is capable of forming at least four hydrogen bridges has the general form (1′) or (2′):

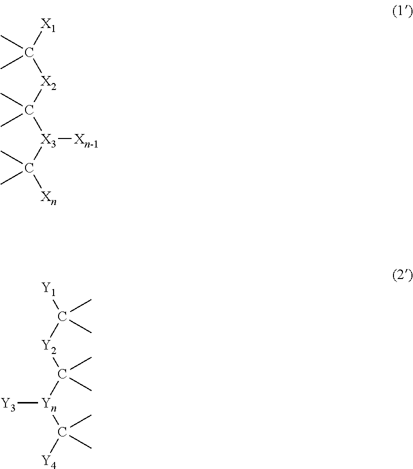

If the structural element (4H) is capable of forming four hydrogen bridges which is preferred according to the invention, the structural element (4H) has preferably the general form (1) or (2):

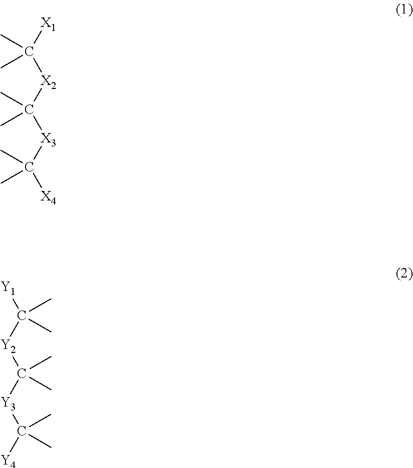

In all general forms shown above the C-X; and C-Y; linkages each represent a single or double bond, n is 4 or more and Xi. X represent donors or acceptors that form hydrogen bridges with the H-bridge-forming unit containing a corresponding structural element (2) linked to them, with Xi representing a donor and Yi an acceptor or vice versa. Properties of the structural element having general forms (1′), (2′), (1) or (2) are disclosed in U.S. Pat. No. 6,320,018 and is incorporated herein by reference.

The structural elements (4H) have at least four donors and/or acceptors, preferably four donors and/or acceptors, so that they may form at least four hydrogen bridges in pairs with each another. Preferably the structural elements (4H) have at least two successive donors, followed by at least two acceptors, preferably two successive donors followed by two successive acceptors, preferably structural elements according to general form (1′) or more preferably (1) with n=4, in which X₁ and X₂ represent a donor and an acceptor, respectively, and X₃ and X₄ represent an acceptor and a donor, respectively. According to the invention, the donors and acceptors are preferably O, S, and N atoms.

Molecules that can be used to construct the structural element (4H) are preferably nitrogen containing compounds that are reacted with isocyanates, thioisocyanates or activated amines, or that are activated and reacted with primary amines, to obtain a urea or thiourea moiety that is part of the quadruple hydrogen bonding site. The nitrogen containing compound is preferably an isocytosine derivative (i. e. a 2-amino-4-hydroxy-pyrimidine derivative) or a triazine derivative, or a tautomer and/or enantiomer of these derivatives. More preferably, the nitrogen containing compound is an isocytosine derivative having a proton or aliphatic-substituent containing a functional group in the 5-position and an alkyl-substituent in the 6-position, most preferably 2-hydroxy-ethyl or propionic acid ester in the 5-position and methyl in the 6-position, or hydrogen in the 5-position and methyl in the 6-position. The isocyanates or the thioisocyanates can be monofunctional isocyanates or monofunctional thioisocyanates or bifunctional diisocyanates or bifunctional thioisocyanates (for example alkyl or aryl (di) (thio) isocyanate (s)).

According to the invention, a subunit that comprises the structural element 4H is particularly suitably represented in the compounds having the general formulae (3) or (4), and tautomers and/or enantiomers thereof (see below). A subunit that comprises a precursor of the structural element 4H, denoted 4H*, is particularly suitably represented in the compounds having the general formulae (5) or (6). The X in formulae (4) and (6) is preferably a nitrogen atom, but it may also be a carbon atom with attached R₄-group.

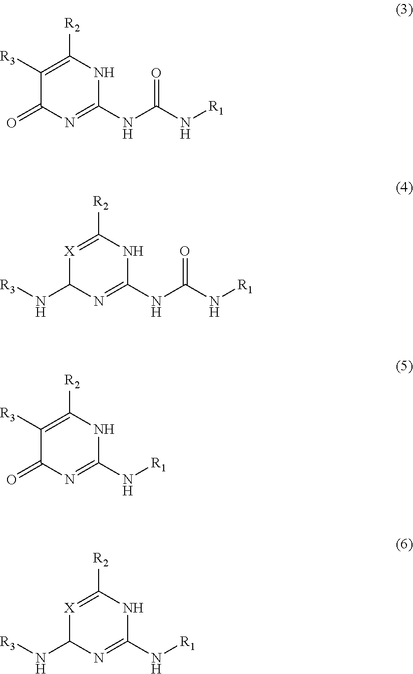

R₁ is a direct bond connecting the 4H unit to the linker L. R₂, R₃ and R₄ may be hydrogen; C₁₋₂₄ alkyl; C₆₋₁₂ aryl; C₁₋₂₄ alkyl ether. In preferred embodiments of the present invention and/or embodiments thereof, at least one of R₂, R₃ and R₄ is hydrogen, preferably R₄ is a hydrogen or a C₁₋₁₆alkyl.

In preferred embodiments of the present invention and/or embodiments thereof at least one of R₂ and R₃ is a hydrogen or C₁₋₁₆alkyl, preferably a hydrogen. Preferably R₃ is hydrogen. Preferably R₂, R₃, R₄ is hydrogen or C₁₋₁₆alkyl. Suitable alkyls for R₂, R₃, R₄ are CH₃, C₁₃H₂₇, or CH₂CH(CH₃)C₃H₆CH(C₂H₆).

In preferred embodiments of the present invention and/or embodiments thereof the 4H unit is

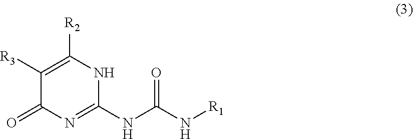

In preferred embodiments of the present invention and/or embodiments thereof the 4H unit is

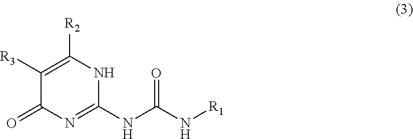

wherein R¹ is a direct bond, and one of R² and R³ is hydrogen.

In preferred embodiments of the present invention and/or embodiments thereof the 4H unit is

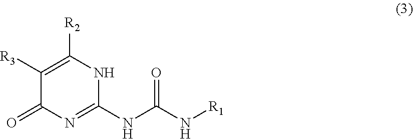

wherein, R¹ is a direct bond, and one of R² and R³ is hydrogen and R² or R³ is an C₁₋₂₄ alkyl.

In preferred embodiments of the present invention and/or embodiments thereof the monofunctional subunit comprises one 4H unit.

In preferred embodiments of the present invention and/or embodiments thereof the bifunctional subunit comprises two 4H units.

The subunit also comprises a linker group L₁ and L₂. L₁ and L₂ may be selected from the group comprising C₁₋₅₀ alkyl, or C₂₋₅₀ alkenyl, or C₂₋₅₀ ether which may linear, or branched, preferably linear. The linker L₁ and L₂ may be substituted with substituents selected from the group comprising OH, CO, CO(O)R₅, C₁₋₆alkyl, C₁₋₆alkyloxy, C₁₋₆alkylthio, C₁₋₆alkylsulfonyl, aminosulfonyl, C₁₋₆alkyloxycarbonyl, C₁₋₆acyl, C₁₋₆acylamino, N(R₅)₂, —CN, —NCO, halo, —NCS, NCS(O)R₅, C(R₅)═NOR₅. NHC(O)R₅, C₅₋₁₂ aryl.

In preferred embodiments of the present invention and/or embodiments thereof, L is a C₁₋₅₀ alkyl, preferably a C₁₋₂₄ alkyl, more preferably a C₁₋₂₀ alkyl, more preferably a C₁₋₁₆ alkyl, more preferably a C₁₋₁₄ alkyl, more preferably a C₁₋₁₂ alkyl. Preferably the alkyl of linker L is linear. Suitably linkers comprise C₄H₈, C₆H₁₂, C₈H₁₄, and/or C₁₂H₂₄. In preferred embodiments of the subunit of the present invention and/or embodiments thereof the linkers may be different or the same. For example linker L₁, is different from the linker L₂, but the two linkers L₁, and L₂ may also be the same. In preferred embodiments the sum of the length of the two linkers L₁ and L₂ does not exceed 80 carbon atoms, preferably does not exceed 70 carbon atoms, more preferably does not exceed 60 carbon atoms, more preferably does not exceed 50 carbon atoms, more preferably does not exceed 45 carbon atoms, more preferably does not exceed 40 carbon atoms, more preferably does not exceed 35 carbon atoms, more preferably does not exceed 30 carbon atoms, more preferably does not exceed 25 carbon atoms, more preferably does not exceed 23 carbon atoms, more preferably does not exceed 20 carbon atoms, more preferably does not exceed 18 carbon atoms, more preferably does not exceed 16 carbon atoms, more preferably does not exceed 15 carbon atoms. In preferred embodiments the sum of the length of the two linkers L₁ and L₂ is between 4 and 50, preferably the sum of the length of the two linkers L₁ and L₂ is between 8 and 45, preferably the sum of the length of the two linkers L₁ and L₂ is between 10 and 40, preferably the sum of the length of the two linkers L₁ and L₂ is between 12 and 38, preferably the sum of the length of the two linkers L₁ and L₂ is between 14 and 35, preferably the sum of the length of the two linkers L₁ and L₂ is between 16 and 32, preferably the sum of the length of the two linkers L₁ and L₂ is between 18 and 30, preferably the sum of the length of the two linkers L₁ and L₂ is between 20 and 28, preferably the sum of the length of the two linkers L₁ and L₂ is between 22 and 26.

The subunit also comprises a functional group F₁ and F₂. F₁ is a hydrogen bonding group and is —NH—C(═O)—NH and F₂ is a functional group consisting of —NRₐ—C(X)—NRₐ— or —NRₐ—C(X)—X—, wherein X is O or S, preferably O, and wherein Rₐ is hydrogen, or C₁₋₁₂ alkyl. The C₁₋₁₂ alkyl may be linear or branched and/or may be substituted. In preferred embodiments of the present invention and/or embodiments thereof F₂ is selected from the group consisting of —NRₐ—C(═O)—NRₐ—, and NRₐ—C(═O)—O—, more preferably selected from the group consisting of —NH—C(═O)—NH—, and —NH—C(═O)—O—. In preferred embodiments of the present invention and/or embodiments thereof F₂ is —NRₐ—C(X)—X—, more preferably NRₐ—C(═O)—O—, and most preferably —NH—C(═O)—O—.

The bifunctional subunit with formula (II) comprises a polyethyleneglycol linker G. G is a polyethyleneglycol linker with a molecular weight of at least 500 Dalton. In preferred embodiments the linker G has a molecular weight between 1 and 100 kD, more preferably between 2 and 75 kD, more preferably between 3 and 60 kD, more preferably between 4 and 50 more preferably between, more preferably between 5 and 45 kD, more preferably between 6 and 40 kD, more preferably between 7 and 35 kD, more preferably between 8 and 30 kD, more preferably between 9 and 25 kD, more preferably between 10 and 22 kD, more preferably between 12 and 20 kD, more preferably between 14 and 18 kD, more preferably between 15 and 16 kD.

The monofunctional subunit (I) comprises a polyethyleneglycol linker P. Linker P is a linker with 0 to 1000 ethylene glycol monomers. Preferably linker P consists of 0 to 800 monomers, more preferably of 1 to 700 monomers, more preferably of 2 to 500 monomers, more preferably of 3 to 450 monomers, more preferably of 4 to 400 monomers, more preferably of 5 to 350 monomers, more preferably of 6 to 300 monomers, more preferably of 7 to 250 monomers, more preferably of 8 to 225 monomers, more preferably of 9 to 200 monomers, more preferably of 10 to 180 monomers, more preferably of 11 to 160 monomers, more preferably of 12 to 150 monomers, more preferably of 13 to 140 monomers, more preferably of 14 to 130 monomers, more preferably of 15 to 120 monomers, more preferably of 16 to 110 monomers, more preferably of 17 to 100 monomers, more preferably of 18 to 90 monomers, more preferably of 19 to 80 monomers, more preferably of 20 to 70 monomers.

The subunit also comprises a linker E. E may be a direct bond, linker L_{E}, linker P_{E}, or combinations of L_{E} and P_{E} linkers. L_{E} is a linker as defined with L₁ or L₂. P_{E} is a polyethyleneglycol linker as defined with polyethyleneglycol linker P. It is thus envisioned that E may be combinations of linker L_{E} and P_{E}, such as C₁₋₅₀ alkyl linked to a polyethyleneglycol linker with 0 to 1000 monomers. Also several combinations are possible of several L_{E} and P_{E} linkers. Combinations such as L_{E}-P_{E}-, L_{E}-P_{E}-L_{E}, P_{E}-L_{E}, P_{E}-L_{E}-P_{E}, L_{E}-P_{E}-L_{E}-P_{E}, P_{E}-L_{E}-P_{E}-L_{E}, L_{E}-P_{E}-L_{E}- P_{E}-L_{E}, P_{E}-L_{E}-P_{E}-L_{E}-P_{E} and combinations thereof and other combinations are possible and within the scope of the invention. A skilled person is well able to find a suitable E linker with L_{E} and P_{E} or with a combination of one or more L_{E} and P_{E} linkers. The L_{E} and P_{E} linkers in E may be the same or may be different. The L_{E} and P_{E} linkers in E may be the same or may be different from L₁, L₂, and P. In preferred embodiments the sum of the length of the linkers L₁, L₂, P, L_{E} and P_{E} does not exceed 2100 carbon atoms, preferably does not exceed 2000 carbon atoms, more preferably does not exceed 1800 carbon atoms, more preferably does not exceed 1500 carbon atoms, more preferably does not exceed 1200 carbon atoms, more preferably does not exceed 1000 carbon atoms, more preferably does not exceed 800 carbon atoms, more preferably does not exceed 500 carbon atoms, more preferably does not exceed 300 carbon atoms, more preferably does not exceed 250 carbon atoms, more preferably does not exceed 200 carbon atoms, more preferably does not exceed 180 carbon atoms, more preferably does not exceed 160 carbon atoms, more preferably does not exceed 150 carbon atoms.

In preferred embodiments the sum of the length of the linkers L₁, L₂, and L_{E} is between 4 and 80, preferably the sum of the length of the linkers L₁, L₂, and L_{E} is between 8 and 60, preferably the sum of the length of the linkers L₁, L₂, and L_{E} is between 10 and 50, preferably the sum of the length of the linkers L₁, L₂, and L_{E} is between 12 and 45, preferably the sum of the length of the linkers L₁, L₂, and L_{E} is between 14 and 40, preferably the sum of the length of the linkers L₁, L₂, and L_{E} is between 16 and 35, preferably the sum of the length of the linkers L₁, L₂, and L_{E} is between 18 and 32, preferably the sum of the length of the linkers L₁, L₂, and L_{E} is between 20 and 30, preferably the sum of the length of the linkers L₁, L₂, and L_{E} is between 22 and 26.

Preferably the length of the linkers P and P_{E} is between 0 and 800 monomers, more preferably between 1 and 700 monomers, more preferably between 2 and 500 monomers, more preferably between 3 and 450 monomers, more preferably between 4 and 400 monomers, more preferably between 5 and 350 monomers, more preferably between 6 and 300 monomers, more preferably between 7 and 250 monomers, more preferably between 8 and 225 monomers, more preferably between 9 and 200 monomers, more preferably between 10 and 180 monomers, more preferably between 11 and 160 monomers, more preferably between 12 and 150 monomers, more preferably between 13 and 140 monomers, more preferably between 14 and 130 monomers, more preferably between 15 and 120 monomers, more preferably between 16 and 110 monomers, more preferably between 17 and 100 monomers, more preferably between 18 and 90 monomers, more preferably between 19 and 80 monomers, more preferably between 20 and 70 monomers.

The monofunctional subunit (I) also comprises a functional group Z. The functional group determines the property of the supramolecular particle. The functional group Z may be selected from the group comprising a neutral moiety, ionic moiety, peptide, therapeutic moiety, imaging agent, fluorescent moiety, targeting moiety, endosomal escape agent moiety, cell-penetrating peptides, antigen, adjuvant, and/or antibody. Preferably the functional group Z is not a polymer. Preferably the functional group Z is not a 4H unit. Preferably Z is not a UPy moiety or a functional group comprising a UPy moiety.

In preferred embodiments of the invention and/or embodiments thereof, the functional group Z is a ionic moiety. In preferred embodiments of the invention and/or embodiments thereof, the functional group Z is a cationic moiety.

The ionic moiety is a charged group or a ionogenic group that is a precursor of a ionic group and that may be converted into a ionic group. The ionic group may be cationic or anionic. Suitable ionogenic groups are for example (tertiary) amine, pyridine, carboxylic acid or carboxylic ester groups whereas suitable ionic groups are for example quarternary amine (ammonium derivatives which may be linear, branched or cyclic including compounds having a nitrogen atom in the ring, e.g. piperidinium), pyridinium, carboxylate, sulfonate and phosphate groups. Suitable ionic groups may also be charged amino acids and or charged peptides, or peptides wherein part of the amino acids are charged amino acids, peptides with a net charge. Conversion from an ionogenic group to an ionic group is typically achieved by protonation or deprotonation. Alternatively, conversion is achieved by alkylation or saponification of the ionogenic group.

Preferably, the ionic groups are selected from the groups that are derived from —N⁺(R₆)₃X⁻¹, —S(O)OH; —S(O)₂OH; —P(O)(R₆)(OH); —P(O)(OH)₂, wherein R₆ is independently selected from the group consisting of hydrogen, hydroxy, linear, branched or cyclic C₁₋₆ alkyl groups, C₆₋₁₆ aryl groups, and wherein X is the counter ion. Moieties comprising one or more nitrogen atoms can be used to obtain cationic moieties. Cationic comprising one or more nitrogen atom that can be used, are, for example, compounds of the following general molecular formulae:

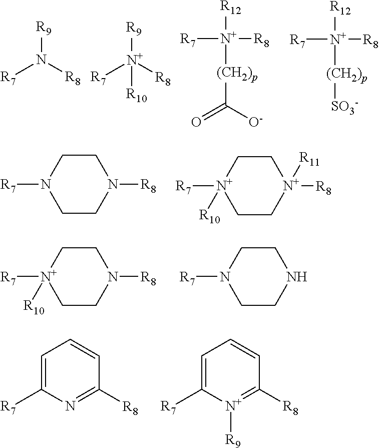

in which R₇ and R₅ are independently selected from the group consisting of linear, branched or cyclic C₂₋₈ alkyl groups, R₉, R₁₀, and R₁₁ are independently selected from the group consisting of linear or branched C₁₋₆ alkyl groups, aryl groups or (C₁₋₄)alkyl aryl groups, and R₁₂ is selected from the group consisting of H, linear or branched C₁₋₆ alkyl groups, aryl group or (C₁₋₄)alkyl arylgroup. Aryl is preferably phenyl. In preferred embodiments p is 1, 2 or 3.

X may be any counter anion, but is preferably a chloride, bromide, iodide, carboxylate, phosphate PO₄³⁻, sulfate SO₄²⁻, C₁₋₆alkyl sulfate, C₁₋₆ alkyl phosphate or C₁₋₆ carboxylate.

Functional Z groups comprising sulfonate, phosphate or carboxylate groups can be used to obtain anionic subunits. Functional Z group with sulfonate or carboxylate groups that can be used, are, for example, 2,2-bis(hydroxymethyl)-propionic acid, or compounds of the general formulae:

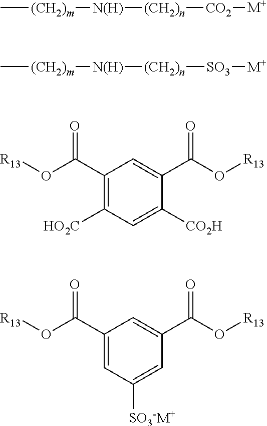

in which m and n are, independently an integer from 1 to 8, in particular from 1 to 6, M⁺ represents a metal cation with any positive charge (i.e. 1+, 2+, 3+, 4+ etc.), preferably a cation derived from an alkaline metal or an alkaline earth metal, more preferably Li⁺, Na⁺, or K⁺, R₁₃ is preferably a C₂₋₁₈ linear, branched or cyclic alkylene group.

In preferred embodiments of this invention, the ionic moiety is selected from a group consisting of NH₃⁺, N⁺-methyl-di-2-ethanolamine, 2,6-bis-(hydroxymethyl)-pyridine, 2,2-bis(hydroxymethyl)-propionic acid, or diesters of diols with the alkali salt of 5-sulfo isophthalic acid. More preferably, the ionic moiety is NH₃⁺, N⁺-methyl-diethanolamine, 2,6-bis-(hydroxymethyl)-pyridine or 2,2-bis(hydroxymethyl)-propionic acid, most preferably the ionic moiety is NH₃⁺, N⁺-methyl-diethanolamine.

Cationic particles are particles that comprise at least one cationic subunit. Cationic subunits are monofunctional subunits that comprise a cationic Z group. Cationic subunits are suitable for binding negatively charged molecules such as nucleic acids. Particles wherein at least a part of the subunits are cationic are then able to bind negatively charged molecules such a nucleic acids. Such particles wherein at least part of the subunits are cationic are able to retain negatively charged molecules. Experiments show that particles with 50% cationic subunits and 50% neutral subunits are able to retain RNA molecules. In preferred embodiments of particles of the present invention and/or embodiments thereof at least 1% of the subunits comprises a cationic Z moiety, more preferably at least 2% of the subunits comprises a cationic Z moiety, at least 5% of the subunits comprises a cationic Z moiety, more preferably at least 7% of the subunits comprises a cationic Z moiety, more preferably at least 10% of the subunits comprises a cationic Z moiety, more preferably at least 12% of the subunits comprises a cationic Z moiety, more preferably at least 15% of the subunits comprises a cationic Z moiety, more preferably at least 20% of the subunits comprises a cationic Z moiety, more preferably at least 25% of the subunits comprises a cationic Z moiety, more preferably at least 30% of the subunits comprises a cationic Z moiety, more preferably at least 35% of the subunits comprises a cationic Z moiety, more preferably at least 40% of the subunits comprises a cationic Z moiety, more preferably at least 45% of the subunits comprises a cationic Z moiety, more preferably at least 50% of the subunits comprises a cationic Z moiety, more preferably at least 60% of the subunits comprises a cationic Z moiety, more preferably at least 70% of the subunits comprises a cationic Z moiety, more preferably at least 80% of the subunits comprises a cationic Z moiety, more preferably at least 90% of the subunits comprises a cationic Z moiety.

Depending on the function of the particle a skilled person will be able based on the information contained herein and common general knowledge determine the amount of cationic subunits in the particles. Suitably the particles comprise between 20% and 80% of cationic subunits, more suitably between 30% and 70% of cationic subunits, more suitably between 40% and 60% of cationic subunits and more suitably between 45% and 55% of cationic subunits. A very suitable particle comprises about 50%-60% of cationic subunits. Preferred particles are particles where the N/P ratio is 2-40, more preferably the N/P ratio is between 3-35, more preferably the N/P ratio is between 4-30, more preferably the N/P ratio is between 5-25, more preferably the N/P ratio is between 6-20, more preferably the N/P ratio is between 7-18, more preferably the N/P ratio is between 8-15, more preferably the N/P ratio is between 9-12. The N/P ratio is the ratio of end amine groups in the particle (N) and the phosphate groups of a nucleic acid backbone (P).

It was also seen that particle wherein at least a part of the subunits were cationic were able to bind to the surface of the cell. Without wishing to be bound to theory, the binding of the at least partly cationic particles is probably due to electrostatic forces between the negatively charged cell membrane and the positively charged cationic particles. Cationic particles are particles wherein at least part of the subunits are cationic subunits. It was also seen that cationic particles are able to internalize into cells. In preferred embodiment the particles have a Z-potential of between 0 and +100V, more preferably between 1V and +75V, more preferably between 2V and +60V, more preferably between 3V and +50V, more preferably between 4V and +45V, more preferably between 5V and +40V, more preferably between 6V and +35V, more preferably between 7V and +30V, more preferably between 8V and +25V, more preferably between 9V and +20V, more preferably between 10V and +18V, more preferably between 12V and +16V.

Anionic subunits are monofunctional subunits that comprise a anionic Z group. Anionic subunits are suitable for binding positively charged molecules such as positively charged peptides, positively charged drugs, positively charged dyes, positively charged targeting compounds, positively charged markers. Particles wherein at least a part of the subunits are anionic are then able to bind positively charged molecules. Such particles wherein at least part of the subunits are anionic are able to retain positively charged molecules. In preferred embodiments of particles of the present invention and/or embodiments thereof at least 10% of the subunits comprises a anionic Z moiety, more preferably at least 20% of the subunits comprises a anionic Z moiety, more preferably at least 25% of the subunits comprises a anionic Z moiety, more preferably at least 30% of the subunits comprises a anionic Z moiety, more preferably at least 35% of the subunits comprises a anionic Z moiety, more preferably at least 40% of the subunits comprises a anionic Z moiety, more preferably at least 50% of the subunits comprises a anionic Z moiety, more preferably at least 70% of the subunits comprises a anionic Z moiety, more preferably at least 90% of the subunits comprises a anionic Z moiety.

Depending on the function of the particle a skilled person will be able based on the information contained herein and common general knowledge determine the amount of anionic subunits in the particles. Suitably the particles comprise between 20% and 80% of anionic subunits, more suitably between 30% and 70% of anionic subunits, more suitably between 40% and 60% of anionic subunits and more suitably between 45% and 55% of anionic subunits.

In preferred embodiments of the invention, it is envisioned that particles may contain both cationic and anionic subunits, also in addition to other subunits such as neutral subunits or subunits with other functional Z groups. In such a way both a positively charged molecule and a negatively charged molecule may be incorporated in the particle.

The functional Z group may also be a neutral moiety. The neutral moiety is a neutral group carrying no charge. It may be non-polar substituents, such as alkyls, and substituted alkyls. The neutral moiety may be selected from the group comprising OH, —NR_{b}R_{c}, NRₐC(═O)R_{d} C₁₋₁₂alkoxy, C₁₋₁₂alkyl, C₂₋₁₂-alkenyl, C₂₋₁₂alkyl ether CO(O)R_{d}, —CN, —NCS(O)R_{d}, C(R_{b})═NOR_{d}. NHC(O)R_{d}. Rₐ is hydrogen, or C₁₋₁₂ alkyl. The C₁₋₁₂ alkyl may be linear or branched and/or may be substituted. Preferably Rₐ is hydrogen or C₁₋₆, more preferably, hydrogen, methyl, ethyl, or propyl. R_{d} is C₁₋₁₂ alkyl, the C₁₋₁₂ alkyl may be linear or branched and/or may be substituted. Preferably R_{d} is C₁₋₆, more preferably, methyl, ethyl, or propyl.

R_{b} and R_{c} are each independently hydrogen C₁₋₁₂alkoxy, C₁₋₁₂alkyl, C₁₋₁₂-alkenyl, C₂₋₁₂alkyl ether. Preferably R_{b} and R_{c} are methyl, ethyl and propyl. In a preferred embodiment if one of R_{b} or R_{c} is hydrogen the other is not hydrogen. In a preferred embodiment, R_{b} and R_{c}, are not hydrogen. The alkyl, alkenyl, alkynyl and/or alkyl ether may comprise heteroatoms such as N, O and/or S, preferably N or O, preferably N, preferably O. The alkyls may be substituted with uncharged groups such as OH, C₁₋₆alkoxy, C₁₋₆alkyl, C₁₋₆-alkenyl, C₂₋₆alkyl ether and —NRₐR_{b}. In preferred embodiments the neutral moiety is hydroxyl, —NR_{b}R_{c}, methoxy, ethoxy, propoxy, methyl, ethyl. Suitable neutral moieties are selected from the group consisting of NRₐC(═O)R_{d}. It was found that supramolecular complexes with subunits wherein Z is a neutral moiety comprise hydrophobic pockets wherein compounds may be encapsulated. The more subunits with neutral moieties the more compounds may be encapsulated. The examples show that complexes consisting of 100% neutral subunits is able to encapsulate the dye Nile Red very effectively giving the maximum fluorescent intensity. Increasing amounts of neutral subunits in the particles increases the amount of Nile Red which is encapsulated.

It is to be understood that when neutral subunits is mentioned, subunits wherein the Z contains a neutral moiety is meant. Supramolecular complexes wherein at least a part of the subunits comprise a neutral moiety for Z are thus very suitable for encapsulating compounds, especially hydrophobic compounds, therapeutic compounds such as hydrophobic drugs. A neutral subunit is a monofunctional subunit wherein Z is a neutral moiety. In preferred embodiments of particles of the present invention and/or embodiments thereof at least 10% of the subunits is a neutral subunit, more preferably at least 20% of the subunits is a neutral subunit, more preferably at least 25% of the subunits is a neutral subunit, more preferably at least 30% of the subunits is a neutral subunit, more preferably at least 35% of the subunits is a neutral subunit, more preferably at least 40% of the subunits is a neutral subunit, more preferably at least 50% of the subunits is a neutral subunit, more preferably at least 70% of the subunits is a neutral subunit, more preferably at least 90% of the subunits is a neutral subunit. It was also found that particles comprising neutral subunits may be used to target cells. Particles with 50% neutral subunits and 50% cationic subunits are able to bind to cells and internalise into the cells. In preferred embodiments the particle of the present invention and/or embodiments thereof comprise less than 80% neutral subunits, more preferably less than 70% neutral subunits, more preferably less than 60% neutral subunits, more preferably less than 50% neutral subunits, more preferably less than 40% neutral subunits, more preferably less than 30% neutral subunits, more preferably less than 20% neutral subunits, more preferably less than 10% neutral subunits. Depending on the function of the particle a skilled person will be able based on the information contained herein determine the amount of neutral subunits in the particles. Suitably the particles comprise between 20% and 80% of neutral subunits, more suitably between 30% and 70% of neutral subunits, more suitably between 40% and 60% of neutral subunits and more suitably between 45% and 55% of neutral subunits. A very suitable particle comprises about 50%-60% of neutral subunits.

The functional Z group may also be a peptide. Peptides are molecule comprising amino acids connected to each other with peptide bonds. Peptide generally have an amino terminus (also referred to as N-terminus or amino terminal amino acid), a carboxyl terminus (also referred to as C-terminus terminal carboxyl terminal amino acid) and internal amino acids located between the amino terminus and the carboxyl terminus. According to the invention a peptide may be up to 1000 amino acids long, e.g. between 10 and 500 amino acids, preferably between 12 and 450 amino acids, more preferably between 15 and 400 amino acids, more preferably between 17 and 375 amino acids, more preferably between 20 and 350 amino acids, more preferably between 22 and 300 amino acids, more preferably between 25 and 250 amino acids, more preferably between 27 and 225 amino acids, more preferably between 30 and 200 amino acids, more preferably between 33 and 175 amino acids, more preferably between 35 and 150 amino acids, more preferably between 37 and 150 amino acids, more preferably between 40 and 125 amino acids, more preferably between 45 and 100 amino acids, more preferably between 50 and 85 amino acids, more preferably between 55 and 75 amino acids and most preferably between 60 and 70 amino acids. Suitable peptides comprise 3 to 100 amino acids, more preferably 3 to 90 amino acids, more preferably 3 to 80 amino acids and most preferable from 3 to 70 amino acids. Peptides have an amino end and a carboxyl end, unless they are cyclic peptides. It is to be understood that polypeptides, oligopeptides and even proteins are envisioned under the term peptides according to the present invention.

Preferably the peptide is a peptide having a function selected from the group consisting of targeting, therapeutic, cell-entry. A targeting peptide is a peptide is able to target a specific location such as specific tissue, specific cell type. A targeting peptide may be a peptide is able to bind to receptors that are present in specific tissue or on specific cells. Therapeutic peptides have a therapeutic activity, directed toward healing or curing a biological disorder. Examples of said therapeutic peptides include pituitary hormones such as vasopressin, oxytocin, melanocyte stimulating hormones, adrenocorticotropic hormones, growth hormones; hypothalamic hormones such as growth hormone releasing factor, corticotropin releasing factor, prolactin releasing peptides, gonadotropin releasing hormone and its associated peptides, luteinizing hormone release hormones, thyrotropin releasing hormone, orexin, and somatostatin; thyroid hormones such as calcitonins, calcitonin precursors, and calcitonin gene related peptides; parathyroid hormones and their related proteins; pancreatic hormones such as insulin and insulin-like peptides, glucagon, somatostatin, pancreatic polypeptides, amylin, peptide YY, and neuropeptide Y; digestive hormones such as gastrin, gastrin releasing peptides, gastrin inhibitory peptides, cholecystokinin, secretin, motilin, and vasoactive intestinal peptide; natriuretic peptides such as atrial natriuretic peptides, brain natriuretic peptides, and C-type natriuretic peptides; neurokinins such as neurokinin A, neurokinin B, and substance P; renin related peptides such as renin substrates and inhibitors and angiotensins; endothelins, including big endothelin, endothelin A receptor antagonists, and sarafotoxin peptides; and other peptides such as adrenomedullin peptides, allatostatin peptides, amyloid beta protein fragments, antibiotic and antimicrobial peptides, apoptosis related peptides, bag cell peptides, bombesin, bone Gla protein peptides, CART peptides, chemotactic peptides, cortistatin peptides, fibronectin fragments and fibrin related peptides, FMRF and analog peptides, galanin and related peptides, growth factors and related peptides, Gtherapeutic peptide-binding protein fragments, guanylin and uroguanylin, inhibin peptides, interleukin and interleukin receptor proteins, laminin fragments, leptin fragment peptides, leucokinins, mast cell degranulating peptides, pituitary adenylate cyclase activating polypeptides, pancreastatin, peptide T, polypeptides, virus related peptides, signal transduction reagents, toxins, and miscellaneous peptides such as adjuvant peptide analogs, alpha mating factor, antiarrhythmic peptide, antifreeze polypeptide, anorexigenic peptide, bovine pineal antireproductive peptide, bursin, C3 peptide P16, tumor necrosis factor, cadherin peptide, chromogranin A fragment, contraceptive tetrapeptide, conantokin G, conantokin T, crustacean cardioactive peptide, C-telopeptide, cytochrome b588 peptide, decorsin, delicioius peptide, delta-sleep-inducing peptide, diazempam-binding inhibitor fragment, nitric oxide synthase blocking peptide, OVA peptide, platelet calpain inhibitor (P1), plasminogen activator inhibitor 1, rigin, schizophrenia related peptide, serum thymic factor, sodium potassium Atherapeutic peptidease inhibiro-1, speract, sperm activating peptide, systemin, thrombin receptor agonist, thymic humoral gamma2 factor, thymopentin, thymosin alpha 1, thymus factor, tuftsin, adipokinetic hormone, uremic pentapeptide and other therapeutic peptides The present invention includes peptides which are derivable from the naturally occurring sequence of the peptide. A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence. Included within the scope of the present invention are those molecules which are said to be “derivatives” of a peptide. Such a “derivative” has the following characteristics: (1) it shares substantial homology with the therapeutic peptide or a similarly sized fragment of the peptide and (2) it is capable of functioning with the same therapeutic activity as the peptide.

A derivative of a peptide is said to share “substantial homology” with the peptide if the amino acid sequences of the derivative is at least 80%, and more preferably at least 90%, and most preferably at least 95%, the same as that of either the peptide or a fragment of the peptide having the same number of amino acid residues as the derivative.

The derivatives of the present invention and/or embodiments thereof include fragments which, in addition to containing a sequence that is substantially homologous to that of a naturally occurring peptide may contain one or more additional amino acids at their amino and/or their carboxy termini. Thus, the invention pertains to polypeptide fragments of peptides that may contain one or more amino acids that may not be present in a naturally occurring therapeutic peptide sequence provided that such fragments have a therapeutic activity which exceeds that of the therapeutic peptide. Similarly, the invention includes polypeptide fragments which, although containing a sequence that is substantially homologous to that of a naturally occurring therapeutic peptide, may lack one or more additional amino acids at their amino and/or their carboxy termini that are naturally found on the therapeutic peptide.

Thus, the invention and/or embodiments thereof pertains to polypeptide fragments of peptides that may lack one or more amino acids that are normally present in the naturally occurring peptide sequence, preferably the derivatives of peptide have the same activity or an activity that exceeds that of the original peptide.

The invention and/or embodiments thereof also encompasses the obvious or trivial variants of the above-described fragments which have inconsequential amino acid substitutions (and thus have amino acid sequences which differ from that of the natural sequence) provided that such variants have an activity which is substantially identical to that of the original or derivatives. Examples of obvious or trivial substitutions include the substitution of one basic residue for another (i.e. Arg for Lys), the substitution of one hydrophobic residue for another (i.e. Leu for He), or the substitution of one aromatic residue for another (i.e. Phe for Tyr), etc. As is known in the art, the amino acid residues may be in their protected or unprotected form, using appropriate amino or carboxyl protecting groups as discussed in detail below. The variable length peptides may be in the form of the free amines (on the N-terminus), or acid-addition salts thereof. Common acid addition salts are hydrohalic acid salts, i.e., HBr, HI, or, more preferably, HCI. Useful cations are alkali or alkaline earth metallic cations (i.e., Na, K, Li, Ca, Ba, etc.) or amine cations (i.e., tetraalkylammonium, trialkylammonium, where alkyl can be CιCι₂). Any peptide having a desired activity may be used in this invention. Suitable peptide include cell penetrating peptides, such as TAT peptide, MPG, Pep-1, MAP, fusogenic, antimicrobial peptides (AMPs), bacteriocidal peptides, fungicidal peptides, virucidal peptides,

Cell-penetrating peptides (CPPs) are short peptides that facilitate cellular uptake of the particles of the invention. The particle of the invention is associated with the CPP peptides either through chemical linkage via covalent bonds or through non-covalent interactions. The function of the CPPs are to deliver the particles into cells, a process that commonly occurs through endocytosis with the cargo delivered to the endosomes of living mammalian cells. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake.

An exemplary cell penetrating peptide is the trans-activating transcriptional activator (Tat) from Human Immunodeficiency Virus 1 (HIV-1) could be efficiently taken up from the surrounding media by numerous cell types in culture. Other cell penetrating peptides are MPG, Pep-1, transportan, penetratin, CADY, TP, TP10, arginine octamer. polyarginine sequences, Arg8, VP22 HSV-1 structural protein, SAP Proline-rich motifs, Vectocell® peptides, hCT (9-32), SynB, Pvec, and PPTG1. Cell penetrating peptides may be cationic, essentially containing clusters of polyarginine in their primary sequence or amphipathic. CPPs are generally peptides of less than 30 amino acids, derived from natural or unnatural protein or chimeric sequences.

TABLE 1
Representative CPPs and sequences:

| Peptides | Origin | Sequences |
|---|---|---|
| Peptides | | |
| deriving from | | |
| protein | | |
| transduction | | |
| domains | | |
| Tat | HIV-Tat | PGRKKRRQRRPPQ |
| | protein | |
| Penetratin | Homeodomain | RQIKIWFQNRRMKWKK |
| Transportan | Galanin- | GWTLNSAGYLLGKINLKALAALAKKIL |
| | mastoparan | |
| VP-22 | HSV-1 | DAATATRGRSAASRPTERPRAPAR- |
| | structural | SASRPRRPVD |
| | protein | |
| Amphipathic | | |
| peptides | | |
| MPG | HIV Gp41- | GALFLGFLGAAGSTMGAWSQPKKKRKV |
| | SV40 NLS | |
| Pep-1 | Trp-rich | KETWWETWWTEWSQPKKKRKV |
| | motif-SV40 | |
| | NLS | |
| MAP | Chimeric | KALAKALAKALA |
| SAP | Proline-rich | VRLPPPVRLPPPVRLPPP |
| | motif | |
| PPTG1 | Chimeric | GLFRALLRLLRSLWRLLLRA |
| Other cell- | | |
| penetrating | | |
| peptides: | | |
| cationic | | |
| peptides | | |
| Oligoarginine | Chimeric | Agr8 or Arg9 |
| hCT (9-32) | Human | LGTYTQDFNKTFPQTAIGVGAP |
| | calcitonin | |
| SynB | Protegrin | RGGRLSYSRRRFSTSTGR |
| Pvec | Murine VE- | LLIILRRRIRKQAHAHSK |
| | cadherin | |

In preferred embodiments of particles of the present invention and/or embodiments thereof at least 5% of the monofunctional subunits comprises a peptide, more preferably at least 10% of the monofunctional subunits comprises a peptide, more preferably at least 20% of the monofunctional subunits comprises a peptide, more preferably at least 25% of the monofunctional subunits comprises a peptide, more preferably at least 30% of the monofunctional subunits comprises a peptide, more preferably at least 35% of the monofunctional subunits comprises a peptide, more preferably at least 40% of the monofunctional subunits comprises a peptide, more preferably at least 50% of the monofunctional subunits comprises a peptide, more preferably at least 70% of the monofunctional subunits comprises a peptide, more preferably at least 90% of the monofunctional subunits comprises a peptide.

In preferred embodiments of particles of the present invention and/or embodiments thereof at least 2% of the monofunctional subunits comprises a cell penetrating peptide, more preferably at least 5% of the monofunctional subunits comprises a cell penetrating peptide, more preferably at least 7% of the monofunctional subunits comprises a cell penetrating peptide, more preferably at least 10% of the monofunctional subunits comprises a cell penetrating peptide, more preferably at least 15% of the monofunctional subunits comprises a cell penetrating peptide, more preferably at least 20% of the monofunctional subunits comprises a cell penetrating peptide, more preferably at least 25% of the monofunctional subunits comprises a cell penetrating peptide, more preferably at least 30% of the monofunctional subunits comprises a cell penetrating peptide, more preferably at least 90% of the monofunctional subunits comprises a cell penetrating peptide, more preferably at least 35% of the monofunctional subunits comprises a cell penetrating peptide, more preferably at least 40% of the monofunctional subunits comprises a cell penetrating peptide, more preferably at least 50% of the monofunctional subunits comprises a cell penetrating peptide, more preferably at least 70% of the monofunctional subunits comprises a cell penetrating peptide, more preferably at least 90% of the monofunctional subunits comprises a cell penetrating peptide.

In preferred embodiments of particles of the present invention and/or embodiments thereof at least 2% of the monofunctional subunits comprises a cell targeting peptide, more preferably at least 5% of the monofunctional subunits comprises a cell targeting peptide, more preferably at least 7% of the monofunctional subunits comprises a cell targeting peptide, more preferably at least 10% of the monofunctional subunits comprises a cell targeting peptide, more preferably at least 15% of the monofunctional subunits comprises a cell targeting peptide, more preferably at least 20% of the monofunctional subunits comprises a cell targeting peptide, more preferably at least 25% of the monofunctional subunits comprises a cell targeting peptide, more preferably at least 30% of the monofunctional subunits comprises a cell targeting peptide, more preferably at least 90% of the monofunctional subunits comprises a cell targeting peptide, more preferably at least 35% of the monofunctional subunits comprises a cell targeting peptide, more preferably at least 40% of the monofunctional subunits comprises a cell targeting peptide, more preferably at least 50% of the monofunctional subunits comprises a cell targeting peptide, more preferably at least 70% of the monofunctional subunits comprises a cell targeting peptide, more preferably at least 90% of the monofunctional subunits comprises a cell targeting peptide.

In preferred embodiments of particles of the present invention and/or embodiments thereof at least 2% of the monofunctional subunits comprises a therapeutic peptide, more preferably at least 5% of the monofunctional subunits comprises a therapeutic peptide, more preferably at least 7% of the monofunctional subunits comprises a therapeutic peptide, more preferably at least 10% of the monofunctional subunits comprises a therapeutic peptide, more preferably at least 15% of the monofunctional subunits comprises a therapeutic peptide, more preferably at least 20% of the monofunctional subunits comprises a therapeutic peptide, more preferably at least 25% of the monofunctional subunits comprises a therapeutic peptide, more preferably at least 30% of the monofunctional subunits comprises a therapeutic peptide, more preferably at least 90% of the monofunctional subunits comprises a therapeutic peptide, more preferably at least 35% of the monofunctional subunits comprises a therapeutic peptide, more preferably at least 40% of the monofunctional subunits comprises a therapeutic peptide, more preferably at least 50% of the monofunctional subunits comprises a therapeutic peptide, more preferably at least 70% of the monofunctional subunits comprises a therapeutic peptide, more preferably at least 90% of the monofunctional subunits comprises a therapeutic peptide.

In preferred embodiments of the present invention the particles comprise monofunctional subunits comprising a therapeutic moiety or a therapeutic agent. A therapeutic agent or moiety has an activity directed toward healing or curing a disorder. A therapeutic agent or moiety may be a chemical compound, a peptide, a nucleic acid or an antibody. Antisense oligonucleotide (AON), small-interfering RNA (si-RNA) and micro-RNA (mi-RNA) are suitable therapeutic agents. Cationic subunits are able to bind to the negatively charged phosphate groups of nucleic acids. Alternatively the therapeutic agent, including nucleic acids, may be covalently bound to the subunit. The particles of the present invention may comprise different monofunctional subunits that comprise different therapeutic agents, or monofunctional subunits comprising more than one therapeutic agent. The particles of the invention may also comprise a therapeutic compound encapsulated in the hydrophobic pockets of the particles and in addition, another therapeutic agent bound to at least part of the monofunctional subunits. Suitably particles with therapeutic agents also comprise monofunctional subunits with a targeting moiety, and/or an imaging moiety.

In preferred embodiments of particles of the present invention and/or embodiments thereof at least 10% of the monofunctional subunits comprises a therapeutic moiety, more preferably at least 20% of the monofunctional subunits comprises a therapeutic moiety, more preferably at least 25% of the monofunctional subunits comprises a therapeutic moiety, more preferably at least 30% of the monofunctional subunits comprises a therapeutic moiety, more preferably at least 35% of the monofunctional subunits comprises a therapeutic moiety, more preferably at least 40% of the monofunctional subunits comprises a therapeutic moiety, more preferably at least 50% of the monofunctional subunits comprises a therapeutic moiety, more preferably at least 70% of the monofunctional subunits comprises a therapeutic moiety, more preferably at least 90% of the monofunctional subunits comprises a therapeutic moiety.

Imaging agent or contrast agent may also suitably be used in the present invention and/or embodiments thereof. Many imaging studies such as MRI, PET, CT and x-ray, involve the use of imaging agents. Imaging agents are designed to provide more information about internal organs, cellular processes and tumors, as well as normal tissue. They can be used to diagnose disease as well as monitor treatment effects. Imaging agents may be contrast agents, Quantum dots (QD), Magnetic resonance imaging agents, nuclear medicine imaging agents, PET imaging agents, fluorescent agents, X-ray imaging agents, CT imaging agents, SPECT imaging agents. Quantum dots (QD) represent a relative new class of fluorescent probes that have superior optical properties than classical organic dyes based on fluorescent groups. Quantum dots are colloidal nanocrystals, based on a cadmium-selenium (CdSe) core covered with a zinc-sulfur (ZnS) layer. Magentic resonance imaging moiety may be a metal chelates that increase the contrast signal between normal and diseased tissues by changing the nuclear relaxation times of water molecules in their proximities. Typical examples are gadolinium (Gd³⁺) and low-molecular-weight chelates thereof, and superparamagnetic iron oxide (SPIO). In vivo administration of these agents allows the labelling of tumor cells.

PET and nuclear imaging agents may be ⁶⁴Cu-ATSM: ⁶⁴Cu diacetyl-bis(N⁴-methylthiosemicarbazone), also called ATSM or Copper 64, FDG: ¹⁸F-fluorodeoxyglucose (FDG), ¹⁸F-fluoride and molecules with ¹⁸F fluoride, FLT: 3′-deoxy-3′-[¹⁸F]fluorothymidine (FLT), FMISO: ¹⁸F-fluoromisonidazole, Gallium and compounds comprising Gallium, Technetium-99m and compounds comprising Technetium-99m, Thallium and compounds comprising Thallium. Typical isotopes include ¹¹Carbon, ¹³Nitrogen, ¹⁵oxygen, ¹⁸Fluoride, ⁶⁴Copper, ⁶²Copper, ¹²⁴Iodine, ⁷⁶Bromine, ⁸²Rubenium, ⁶⁸Gallium, with ¹⁸Fluoride the most clinically used.

X-ray and CT imaging agents may be Barium: and Barium containing compounds, Gastrografin, Iodine Contrast Agents.

The imaging agent used in SPECT emits gamma rays, as opposed to the positron emitters (such as ¹⁸F) used in PET. There are a range of radiotracers (such as ^{99m}Technetium, ¹¹¹Indium, ¹²³Iodine, ²⁰¹Tellurium) that can be used, depending on the specific application.

Fluorescent imaging agents and probes are also very suitable for use in the particles of the present invention. Suitable fluorescent imaging agents include Kodak X-SIGHT Dyes and Conjugates, Pz 247, DyLight 750 and 800 Fluors, Cy 5.5 and cy7 Fluors, Alexa Fluor 680 and 750 Dyes, IRDye 680 and 800CW Fluors. Preferred imaging agents that near-infrared fluorphores as they can be used in deeper lying tissue.

A skilled person is able to select appropriate imaging agents to suit his needs. Imaging agents may be found in the molecular imaging and contrast agent database (MICAD) and in the list of FDA approved contrast agents.

Particle of the present invention wherein at least a part of the monofunctional subunits comprise an imaging agent may be used in imaging technique to visualise for example where the particles of the invention are targeted and whether the particles are taken up by cells or not. Suitable particles are particles wherein at least a part of the monofunctional subunits comprises an imaging agent. For example a particle comprising a drug that is encapsulated into the hydrophobic space of the particle, comprising monofunctional subunits with a targeting moiety and comprising monofunctional subunits with a imaging agent may be followed upon administration to see whether and when the particle reach the target tissue, and when the particles are cleared from the body.

In preferred embodiments of particles of the present invention and/or embodiments thereof at least 0.1% of the monofunctional subunits comprises a imaging moiety, more preferably at least 0.5% of the monofunctional subunits comprises a imaging moiety, more preferably at least 1% of the monofunctional subunits comprises a imaging moiety, more preferably at least 2% of the monofunctional subunits comprises a imaging moiety, more preferably at least 3% of the monofunctional subunits comprises a imaging moiety, more preferably at least 4% of the monofunctional subunits comprises a imaging moiety, more preferably at least 5% of the monofunctional subunits comprises a imaging moiety, more preferably at least 7% of the monofunctional subunits comprises a imaging moiety, more preferably at least 10% of the monofunctional subunits comprises a imaging moiety.

In suitable embodiments of the present invention, the particles comprise monofunctional subunits comprising targeting moieties. A targeting moiety is a moiety that targets specific tissues or specific cells or a specific location in a body and direct the particles of the present invention and/or embodiments thereof to predetermined locations. The targeting moiety may bind to a receptor, or antigen, may be able to accumulate in a specific environment such as high or low oxygen, high or low pH, high or low redox environment, hydrophopic environment, or a hydrophilic environment. Targeting moiety may be a peptide, protein, antibody, aptamer, chemical compounds, or ligand. Antibodies recognizing proteins on the surface of target cells, aptamers adapted to target specific proteins, peptides and small molecules able to bind to receptors on the surface of target cells are useful in the present invention. Suitable targeting moieties may be selected from the group comprising RGD containing peptides/protein, hyaluronic acid, somastatin analogues.

The targeting compounds may suitably target dendritic cells (DCs). The human DCs are identified by over expression of human leukocyte antigen (HLA) DR (major histocompatibility complex class II). In addition, the specific markers for identifying the myeloid DCs include CD11c+, CD1a+, BDCA-1+, BDCA-3+, HLA-DR+ whereas for the plamacytoid DCs they are CD11c−, HLADR+, BDCA-2+ and CD123+. In a preferred embodiment, the targeting compound binds or is able to bind to CD11, CD1a, BDCA-1, BDCA-3, HLA-DR, BDCA-2 and CD123, toll-like receptors (TLR), C-type lectin receptors (CLR), and nod-like receptors (NLR). Suitable targeting compounds are presented in table 2.

TABLE 2
targeting compounds:

| Targeting receptor | Targeting compound |
|---|---|
| TLR 1/2 | Pam₃CAG |
| TLR 2/6 | Pam₂CAG |
| TLR 3 | Poly (I:C) |
| TLR 4 | LPS |
| TLR 4 | Lipid A |
| TLR 4 | MPLA |
| TLR 5 | Flagelin |
| TLR 7 | 3M019 |
| TLR 9 | Plasmid DNA |
| TLR 9 | CpG ODN |
| Mincle | TDM |
| Dectin-1 | B-glucan |
| NOD2 | MDP |

In addition, suitable targeting compounds for cancer vaccination may be selected from the group consisting of mannose/mannan, ligands for the Fc receptors for each immunoglobulin class, CD11c/CD18 and DEC 205 receptor targets, DC-SIGN receptor targets. A skilled person is well aware of suitable targeting compounds for desired target cells and is able to select the desired targeting compounds. In the context of this invention, targeting ligand, targeting agent, targeting compound or targeting group are used interchangeably, and all mean a compound that is able to target a specific cell or specific tissue.

Particles with at least a part of the monofunctional subunits wherein the Z is a targeting moiety may be used to specifically deliver therapeutic compounds to the cells and sites of interest. Specific targeting reduces toxicity as the particles of the present invention will preferentially accumulate in tissue of interest and not in other tissue.

In preferred embodiments of particles of the present invention and/or embodiments thereof at least 1% of the monofunctional subunits comprises a targeting moiety, more preferably at least 2% of the monofunctional subunits comprises a targeting moiety, more preferably at least 5% of the monofunctional subunits comprises a targeting moiety, more preferably at least 10% of the monofunctional subunits comprises a targeting moiety, more preferably at least 15% of the monofunctional subunits comprises a targeting moiety, more preferably at least 20% of the monofunctional subunits comprises a targeting moiety, more preferably at least 25% of the monofunctional subunits comprises a targeting moiety, more preferably at least 30% of the monofunctional subunits comprises a targeting moiety, more preferably at least 35% of the monofunctional subunits comprises a targeting moiety.

In suitable embodiments of the present invention and/or embodiments thereof, the particles comprise monofunctional subunits comprising a endosomal escape agent moiety. The endocytic pathway is a major uptake mechanism of cells. Agents taken up by the endocytic pathway become entrapped in endosomes and are degraded by specific enzymes in the lysosome. This may be desired or not desired depending on the purpose. If taken up by the endosomes is not desired, endosomal escape agent may be used. Suitable endosomal escape agents may be chloroquine, TAT peptide, melittin, and mellitin-like peptides, and fusogenic lipid, fusogenic protein. A suitable fusogenic lipid may be dioleoylphosphatidyl-ethanolamine (DOPE). A skilled person is well able to provide fusogenic lipids.

In preferred embodiments of particles of the present invention and/or embodiments thereof at least 10% of the monofunctional subunits comprises a endosomal escape agent, more preferably at least 20% of the monofunctional subunits comprises a endosomal escape agent, more preferably at least 25% of the monofunctional subunits comprises a endosomal escape agent, more preferably at least 30% of the monofunctional subunits comprises a endosomal escape agent, more preferably at least 35% of the monofunctional subunits comprises a endosomal escape agent, more preferably at least 40% of the monofunctional subunits comprises a endosomal escape agent, more preferably at least 50% of the monofunctional subunits comprises a endosomal escape agent, more preferably at least 70% of the monofunctional subunits comprises a endosomal escape agent, more preferably at least 90% of the monofunctional subunits comprises a endosomal escape agent.

In suitable embodiments of the present invention, the particles comprise monofunctional subunits comprising an antigen and/or an immunogen. Antigens are antibody generating compounds. An immunogen is a substance that is able to provoke an immune response. Particles which carry antigens and/or immunogens may be useful in vaccination. Suitable antigens and/or immunogens may be monofunctional subunits from microbes, such as epitopes, (part of) proteins of the outer membrane of microbes and/or tumor cells, toxins, tumor antigens. Suitable particles comprise monofunctional subunits comprising an antigen and/or immunogen and monofunctional subunits comprising a targeting moiety for B and/or T cells.

In preferred embodiments, the antigen may be selected form the group of chemicals, bacteria bacterial excretions such as toxins, LPS, bacteriophages, mycobacterial antigens, ovalbumin, viruses, or any part thereof. Suitably the antigen is a surface protein, or part thereof from bacteria, viruses, bacteriophages, and/or mycobacteria. Suitable examples are antigens from diphtheria toxoid, diphtheria CRM-197, human papillomavirus, malaria virus antigens, west Nile virus, (recombinant) hepatitis A or B (surface or core antigens), cytomegalovirus, HIV, anthrax, rabies, candidiasis, influenza (various type), e.g. subunit like hemagglutinin, and neuraminidase, ortuberculosis, e.g. Ad35-vectored tuberculosis (TB) AERAS-402. The skilled person will be able to select the appropriate antigen based on the type of vaccine and route of administration.

In a preferred embodiment of the present invention and/or embodiments thereof, the particle may comprise more than one antigen. More than one antigen of the same disease agent may be used, and/or antigens from different disease agents may be used for e.g. multivaccines.

In preferred embodiments of particles of the present invention and/or embodiments thereof at least 5% of the monofunctional subunits comprises a antigen and/or immunogen, more preferably at least 10% of the monofunctional subunits comprises a antigen and/or immunogen, more preferably at least 15% of the monofunctional subunits comprises a antigen and/or immunogen, more preferably at least 20% of the monofunctional subunits comprises a antigen and/or immunogen, more preferably at least 25% of the monofunctional subunits comprises a antigen and/or immunogen, more preferably at least 30% of the monofunctional subunits comprises a antigen and/or immunogen, more preferably at least 40% of the monofunctional subunits comprises a antigen and/or immunogen, more preferably at least 50% of the monofunctional subunits comprises a antigen and/or immunogen, more preferably at least 70% of the monofunctional subunits comprises a antigen and/or immunogen.

In suitable embodiments of the present invention, the particles comprise monofunctional subunits comprising an adjuvant. An adjuvant is used to enhance the immune response to an antigen/immunogen. They may be included in a vaccine to enhance the recipient's immune response to the supplied antigen, thus minimizing the amount of foreign material.

Known adjuvants are a very diverse set of compounds ranging from bacterial toxins, particulates, plant derivatives and pathogen-associated molecular patterns (PAMPs). A useful database for potential adjuvants is Vaxjo and may be found on http://www.violinet.org/. The database Vaxjo is hereby incorporated by reference.

Suitable examples of adjuvants may be selected from the group consisting of cobalt oxide, aluminum hydroxide hydrate, aluminumphosphate, potassiumaluminumsulfate, inactivated and dried mycobacteria (usually M. tuberculosis) (part of Freund's adjuvant), CT: Cholera toxin; including CTB: B subunit of cholera toxin, LT: Escherichia coli heat-labile toxin, Imiquimod, Montanide, including Montanide™ ISA51, MF59™: squalene oil, dispersed in the form of 160 nm droplets, conveniently stabilized with a mixture of a high HLB (polysorbate 80) and a low HLB surfactant (sorbitan trioleate), AS02™: squalene and two hydrophobic immune adjuvants, MPL1TM, a synthetic derivative of LPS, and QS-21, a purified saponin plant extract. Preferred adjuvants are alum (aluminium hydroxide), squalene or MF59. In a preferred embodiment of the present invention and/or embodiments thereof, the particle may comprise more than one adjuvant.

Particles with antigens and/or adjuvants of the present invention and/or embodiment thereof may suitably be used for therapeutic and/or prophylactic purposes. Examples of fields of use may be oncology, tuberculosis, bacterial infections, diphtheria, hepatitis B, influenza, HIV, HCV, flavivirus, west-nile virus, dengue virus. It is understood that any kind of indication may be possible, and that the present invention is not limited to the examples indicated above.

In preferred embodiments the particles of the invention and/or embodiments thereof comprise monofunctional subunits with antigens and monofunctional subunits with adjuvants.

In preferred embodiments of particles of the present invention and/or embodiments thereof at least 1% of the monofunctional subunits comprises a adjuvant, more preferably at least 2% of the monofunctional subunits comprises a adjuvant, more preferably at least 5% of the monofunctional subunits comprises a adjuvant, more preferably at least 7% of the monofunctional subunits comprises a adjuvant, more preferably at least 10% of the monofunctional subunits comprises a adjuvant, more preferably at least 15% of the monofunctional subunits comprises a adjuvant, more preferably at least 20% of the monofunctional subunits comprises a adjuvant, more preferably at least 25% of the monofunctional subunits comprises a adjuvant, more preferably at least 30% of the monofunctional subunits comprises a adjuvant.

In suitable embodiments of the present invention, the particles comprise monofunctional subunits comprising an antibody. Any kind or antibody may be used according to the invention. Antibodies may be useful as targeting agents, antigens, and therapeutic agents. A skilled person is able to select the antibody for a specific purpose.

In preferred embodiments of particles of the present invention and/or embodiments thereof at least 5% of the monofunctional subunits comprises a antibody, more preferably at least 7% of the monofunctional subunits comprises a antibody, more preferably at least 10% of the monofunctional subunits comprises a antibody, more preferably at least 15% of the monofunctional subunits comprises a antibody, more preferably at least 20% of the monofunctional subunits comprises a antibody, more preferably at least 25% of the monofunctional subunits comprises a antibody, more preferably at least 30% of the monofunctional subunits comprises a antibody, more preferably at least 40% of the monofunctional subunits comprises a antibody, more preferably at least 50% of the monofunctional subunits comprises a antibody.

It is to be understood that the present invention encompass particles wherein monofunctional subunits are present with different Z group, so that the particles have several functionalities. A skilled person will be able to select the functional group Z, the type of monofunctional subunits depending on the needs of the purpose.

In preferred embodiments of the present invention and/or embodiments thereof the particle comprises dimers of the monofunctional subunits of formula (I), preferably the monofunctional subunits are bonded to each other via the 4H unit.

Different monofunctional subunits, with different 4H groups, different linker L groups, different functional F groups and different functional Z groups may be used to form the supramolecular structure of the particles of the invention. The 4H group of the monofunctional subunits enable the monofunctional subunits to self-assemble in water, by hydrogen bonding and thereby preferably dimerise. The so formed dimers stack upon each other to form supra molecular structures. Mixing in different amount of different functional monofunctional subunits enable the creation of different particles with very many functionalities.

In preferred embodiments of the present invention and/or embodiments thereof the particle comprises at least 10 monofunctional subunits of formula (I). More preferably particle of the present invention and/or embodiments thereof comprise at least, 20 monofunctional subunits, more preferably at least 50 monofunctional subunits, more preferably at least 75 monofunctional subunits, more preferably at least 100 monofunctional subunits, more preferably at least 150 monofunctional subunits, more preferably at least 200 monofunctional subunits, more preferably at least 300 monofunctional subunits, more preferably at least 400 monofunctional subunits, more preferably at least 500 monofunctional subunits, more preferably at least 600 monofunctional subunits, more preferably at least 700 monofunctional subunits, more preferably at least 800 monofunctional subunits, more preferably at least 900 monofunctional subunits, more preferably at least 1000 monofunctional subunits, more preferably at least 1200 monofunctional subunits, more preferably at least 1400 monofunctional subunits, more preferably at least 1600 monofunctional subunits, more preferably at least 1800 monofunctional subunits, more preferably at least 2000 monofunctional subunits, more preferably at least 2500 monofunctional subunits, more preferably at least 3000 monofunctional subunits, more preferably at least 3500 monofunctional subunits, more preferably at least 4000 monofunctional subunits, more preferably at least 4500 monofunctional subunits, more preferably at least 5000 monofunctional subunits. In preferred embodiments of the present invention and/or embodiments thereof the particle comprises between 10 and 5000 monofunctional subunits of formula (I), more preferably between 60 and 4200 monofunctional subunits of formula (I), more preferably between 80 and 3700 monofunctional subunits of formula (I), more preferably between 120 and 3300 monofunctional subunits of formula (I), more preferably between 180 and 2800 monofunctional subunits of formula (I), more preferably between 250 and 2200 monofunctional subunits of formula (I), more preferably between 350 and 1900 monofunctional subunits of formula (I), more preferably between 550 and 1500 monofunctional subunits of formula (I), more preferably between 650 and 1300 monofunctional subunits of formula (I), more preferably between 750 and 1100 monofunctional subunits of formula (I).

In preferred embodiments of the present invention and/or embodiments thereof the particle comprises at least 10 bifunctional subunits of formula (II). More preferably particle of the present invention and/or embodiments thereof comprise at least, 20 bifunctional subunits, more preferably at least 50 bifunctional subunits, more preferably at least 75 bifunctional subunits, more preferably at least 100 bifunctional subunits, more preferably at least 150 bifunctional subunits, more preferably at least 200 bifunctional subunits, more preferably at least 300 bifunctional subunits, more preferably at least 400 bifunctional subunits, more preferably at least 500 bifunctional subunits, more preferably at least 600 bifunctional subunits, more preferably at least 700 bifunctional subunits, more preferably at least 800 bifunctional subunits, more preferably at least 900 bifunctional subunits, more preferably at least 1000 bifunctional subunits, more preferably at least 1200 bifunctional subunits, more preferably at least 1400 bifunctional subunits, more preferably at least 1600 bifunctional subunits, more preferably at least 1800 bifunctional subunits, more preferably at least 2000 bifunctional subunits, more preferably at least 2500 bifunctional subunits, more preferably at least 3000 bifunctional subunits, more preferably at least 3500 bifunctional subunits, more preferably at least 4000 bifunctional subunits, more preferably at least 4500 bifunctional subunits, more preferably at least 5000 bifunctional subunits. In preferred embodiments of the present invention and/or embodiments thereof the particle comprises between 10 and 5000 bifunctional subunits of formula (II), more preferably between 60 and 4200 bifunctional subunits of formula (II), more preferably between 80 and 3700 bifunctional subunits of formula (II), more preferably between 120 and 3300 bifunctional subunits of formula (II), more preferably between 180 and 2800 bifunctional subunits of formula (II), more preferably between 250 and 2200 bifunctional subunits of formula (II), more preferably between 350 and 1900 bifunctional subunits of formula (II), more preferably between 550 and 1500 bifunctional subunits of formula (II), more preferably between 650 and 1300 bifunctional subunits of formula (II), more preferably between 750 and 1100 bifunctional subunits of formula (II).

Suitable particle according to invention and/or embodiments thereof have a hydrodynamic diameter of between 0.2 and 1000 nm, more preferably between 2 and 800 nm, more preferably between 10 and 500 nm, more preferably between 20 and 400 nm, more preferably between 30 and 300 nm, more preferably between 40 and 200 nm, more preferably between 50 and 150 nm, more preferably between 60 and 100 nm, more preferably between 70 and 90 nm, more preferably between 75 and 85 nm.

Suitable particle according to invention and/or embodiments thereof have a dispersity of at least 0.6. The dispersity is a measure of the heterogeneity of sizes of molecules or particles in a mixture and ranges from 0 to 1 wherein 1 indicates a uniform dispersity and a low number indicates much heterogeneity of the sizes of the particles. In preferred embodiments of the invention and/or embodiments thereof the dispersity of the particles is at least 0.65, more preferable at least 0.7, more preferably at least 0.8, more preferably at least 0.85, more preferably at least 0.9, more preferably at least 0.95.

In a very suitable embodiment of the present invention and/or embodiments thereof the monofunctional subunit has formula (III)

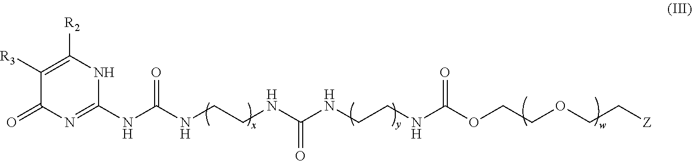

Wherein x is an integer from 1 to 50, y is an integer from 1 to 50, w is an integer from 0 to 1000, z is a functional group selected from the group comprising a neutral moiety, ionic moiety, peptide, therapeutic moiety, imaging agent, fluorescent moiety, targeting moiety, endosomal escape agent, cell-penetrating peptides, antigen, adjuvant, and/or antibody. R₂, and R₃ are hydrogen, C₁₋₂₄ alkyl, C₆₋₁₂ aryl, or C₁₋₂₄ alkyl ether. In a preferred embodiment of the present invention and/or embodiments thereof R₃ is hydrogen. In a preferred embodiment of the present invention and/or embodiments thereof R₂ is a C₁₋₂₄alkyl, preferably CH₃, C₁₃H₂₇, or CH₂CH(CH₃)C₃H₆CH(C₂H₆). In preferred embodiments of the present invention and/or embodiments thereof x is an integer from 1 to 50, more preferably from 2 to 40, more preferably from 3 to 30, more preferably from 4 to 24, more preferably from 5 to 20, more preferably from 6 to 12. In preferred embodiments of the present invention and/or embodiments thereof y is an integer from 1 to 50, more preferably from 2 to 40, more preferably from 3 to 30, more preferably from 4 to 24, more preferably from 5 to 20, more preferably from 6 to 12. In preferred embodiments of the present invention and/or embodiments thereof w is an integer from 0 to 1000, more preferably from 1 to 800, more preferably from 2 to 600, more preferably from 3 to 500, more preferably from 4 to 400, more preferably from 5350, 6-300, 7-250, 8-220, 9-200, 10-190, 11-180, 12 to 170, more preferably from 13 to 160, more preferably from 14 to 150, more preferably from 15- to 45, more preferably from 16 to 140, more preferably from 17 to 135, more preferably from 18 to 130, more preferably from 19 to 125, more preferably from 20 to 120, more preferably from 25 to 115, more preferably from 30 to 110, more preferably from 35 to 110, more preferably from 40 to 95, more preferably from 45 to 90, more preferably from 50 to 85, more preferably from 55 to 80, more preferably from 60 to 75, more preferably from 65 to 70.

Preferred particles of the present invention and/or embodiments thereof comprise at least one monofunctional subunit with formula (I)

4H-L₁-F₁-L₂-F₂—P-E-Z

And at least one bifunctional subunit with formula (II):

4H-L₁-F₁-L₂-F₂-G-F₂-L₂-F₁-L₁-4H (II)

Subunit with formula (II) is a bifunctional subunit at it contains 2 4H units. Because the bifunctional subunit with formula (II) has 2 4H units it is able to cross-link. Under suitable conditions, the bifunctional subunit with formula (II) is able to form a hydrogel.

In a preferred embodiment bifunctional subunit has the following formula (IV):

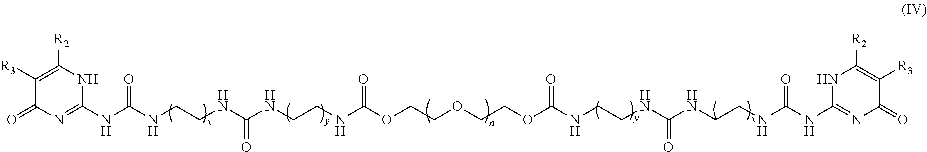

Wherein x is an integer from 1 to 50, y is an integer from 1 to 50, n indicates a linker with a molecular weight of at least 500 dalton, R₂, and R₃ are each independently hydrogen, C₁₋₂₄ alkyl, C₆₋₁₂ aryl, or C₁₋₂₄ alkyl ether.

In a preferred embodiment of the present invention and/or embodiments thereof R₃ is hydrogen. In a preferred embodiment of the present invention and/or embodiments thereof R₂ is a C₁₋₂₄alkyl, preferably CH₃, C₁₃H₂₇, or CH₂CH(CH₃)C₃H₆CH(C₂H₆). In preferred embodiments of the present invention and/or embodiments thereof x is an integer from 1 to 50, more preferably from 2 to 40, more preferably from 3 to 30, more preferably from 4 to 24, more preferably from 5 to 20, more preferably from 6 to 12. In preferred embodiments of the present invention and/or embodiments thereof y is an integer from 1 to 50, more preferably from 2 to 40, more preferably from 3 to 30, more preferably from 4 to 24, more preferably from 5 to 20, more preferably from 6 to 12. n indicates the length of a polyethyleneglycol linker with a molecular weight of at least 500 Dalton. In preferred embodiments n indicates a linker with a molecular weight between 1 and 100 kD, more preferably between 2 and 75 kD, more preferably between 3 and 60 kD, more preferably between 4 and 50 more preferably between, more preferably between 5 and 45 kD, more preferably between 6 and 40 kD, more preferably between 7 and 35 kD, more preferably between 8 and 30 kD, more preferably between 9 and 25 kD, more preferably between 10 and 22 kD, more preferably between 12 and 20 kD, more preferably between 14 and 18 kD, more preferably between 15 and 16 kD. R₂, R₃ is each independently a C₁₋₂₄alkyl, C₂₋₂₄alkenyl, C₂₋₂₄alkynyl, C₃₋₁₂-cycloalkyl; Z is a functional group selected from the group comprising a neutral moiety, ionic moiety, peptide, therapeutic moiety, imaging agent, fluorescent moiety, targeting moiety, endosomal escape agent, cell-penetrating peptides, antigen (e.g. for vaccines), adjuvant, antibody.

In a preferred embodiment of the present invention and/or embodiments thereof, the bifunctional subunit with formula (II) or (IV) is present in at least 2 wt %, more preferably at least 3 wt %, more preferably at least 5 wt %, more preferably at least 7 wt %, more preferably at least 10 wt %, more preferably at least 12 wt %, more preferably at least 15 wt %, more preferably at least 18 wt %, more preferably at least 20 wt %, more preferably at least 22 wt %, more preferably at least 25 wt %.

Particles with at least one bifunctional subunit are able to form a hydrogel. The bifunctional subunit binds to the monofunctional and/or bifunctional subunits of the particle of the invention via hydrogen bonds, making it a supramolecular hydrogel. The supramolecular state enables for control of the sol-gel switching behaviour under mild conditions. The hydrogel of the present invention is pH responsive, enable sol-to-gel switch in a specific pH range. The pH range of the sol-to-gel switch depends on the length of the linker G, and the amount of bifunctional subunit. For example a bifunctional subunit with a 10 kD G linker in an amount of 10 wt % will be fluid at a pH above 9 and a gel at a pH below 8.5. For bifunctional subunits with a 20 kD G linker in an amount of 10 wt % the composition will be fluid at a pH above 10 and fluid a pH below 9.5. A skilled person may easily adjust the amount of bifunctional subunit to tune the pH switchability. Such pH switchability is a very suitable property for example injection. The particle comprising the bifunctional subunit may be injected in a liquid state and become a gel when the pH is changed. The hydrogel further behave liquid like at larger deformations (G′<G″) but recover within minutes when the deformation is removed. The solution of particles of the invention and/or embodiments thereof may have a viscosity of between 0.1 to 5 pa·s, more preferably between 0.2 to 4 pa·s, more preferably between 0.3 to 3 pa·s, more preferably between 0.4 and 2.5 pa·s, more preferably between 0.5 and 2 pa·s, more preferably between 0.6 and 1.8 pa·s, more preferably between 0.7 and 1.6 pa·s, more preferably between 0.8 and 1.4 pa·s, more preferably between 1 and 1.2 pa·s. If the solution is to be injected the viscosity is preferably below 1.2 pa·s, preferably below 1 pa·s, preferably below 0.8 pa·s, more preferably below 0.6 pa·s.

In another aspect the invention is directed to a process for making a particle according to any of the aspects and/or embodiments thereof. The process comprises the steps
i) adding a subunit as defined in the first aspects and/or embodiments thereof to water.

In a specific embodiment of the process for making a particle according to the invention and/or embodiments thereof the invention is directed to a process for making a particle according to the invention and/or embodiments thereof. The process comprises the steps
i) adding to water a monofunctional subunit with formula (I)

4H-L₁-F₁-L₂-F₂—P-E-Z (I)

and/or a bifunctional subunit with formula (II):

4H-L₁-F₁-L₂-F₂-G-F₂-L₂-F₁-L₁-4H (II)

Compounds to be included such as dyes, nucleic acids, drugs, peptides, may be added after the subunit is added to the water or may be added before the subunit is added to the water.

The particles of the present invention and/or embodiment thereof are very suitable for entering a cell. Examples show that cationic particles, e.g. wherein at least a part of the subunits is cationic, are able to enter a cell. In this way compounds such as therapeutic compounds, and imaging agents may be entered into the cell. The particles of the invention thus are very suitable for a research tool, a diagnostic tool or as a therapeutic tool. The particles are also very suitable for labelling cells as they can enter and/or bind the cells. Examples show that cationic particles are able to bind the cells.

The particle according to the present invention and/or embodiments thereof may be used as drug delivery system, or as imaging agent. Therapeutic compounds may either be bound to the subunit or may be encapsulated in the hydrophobic pockets of the supramolecular structure. In a very suitable embodiment, the particle according to the present invention may be used in a prolonged release system, especially if they are in a form of a hydrogel. Another suitable embodiment, the particle according to the present invention may be used in the form of hydrogel as mechanical support for damaged tissue.

Monofunctional subunits with cell penetrating peptides, targeting moieties and/or endosomal escape agent may be used to enhance or add properties to the particles of the invention and/or embodiments thereof. A skilled person is able to choose the subunits depending on the particles need and purpose.

The present invention is further directed to a method of treatment comprising administering the particles of the present invention.

For the purpose of clarity and a concise description features are described herein as part of the same or separate embodiments, however, it will be appreciated that the scope of the invention may include embodiments having combinations of all or some of the features described.

### Examples

### Synthesis of Ureido-Pyrimidinone (UPy)-Molecules

Start with a 1,1′-Carbonyldiimidazole (CDI) activation (1) on a bifunctional poly(ethylene glycol) (PEG) molecule and subsequent coupling with a carboxybenzyl-protected (CBZ-protected) PEG-diamine (2). After deprotection of the CBZ protecting group (3) with triethylsilane a coupling with a UPy-functionalized C6-isocyanate (4) is performed to yield the neutral UPy-C6-C12-PEG11. To yield the amine-functionalized subunit the synthesis is started with a tert-butyloxycarbonyl (BOC)-protected amine on the PEG instead of a methoxy group.

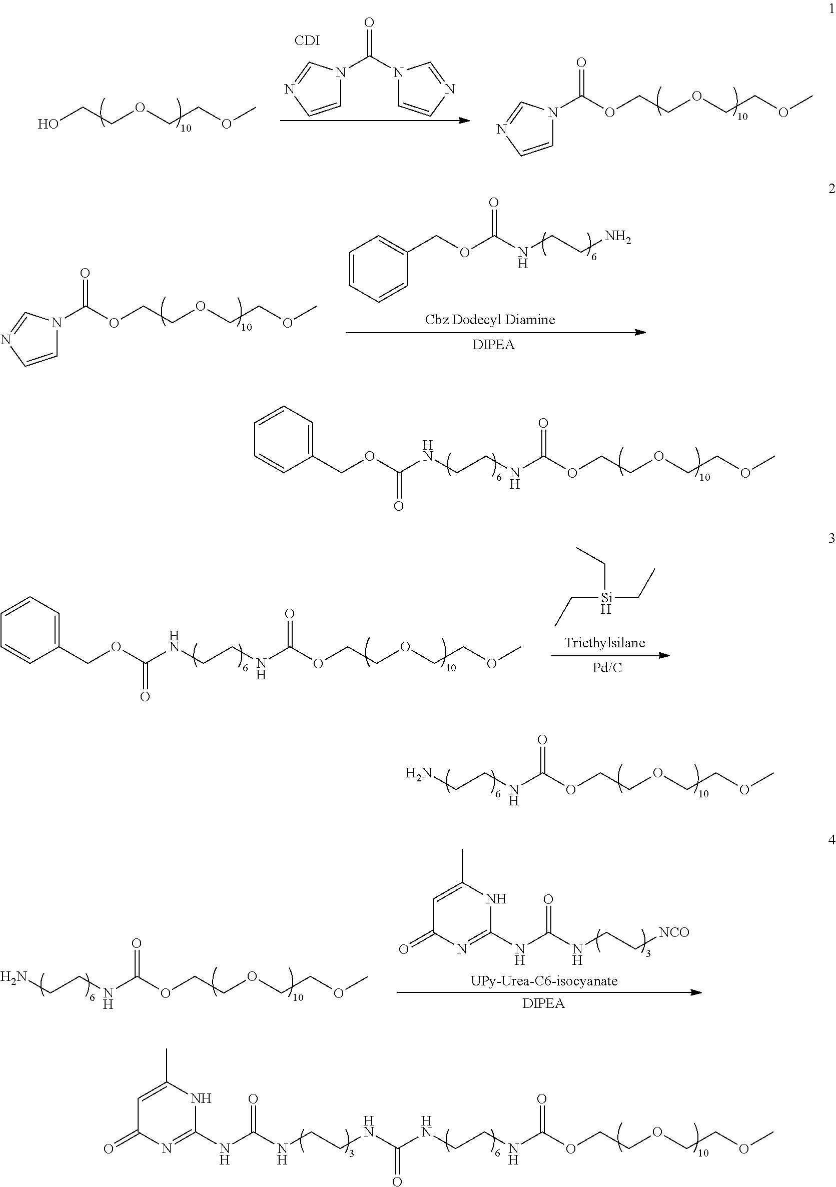

In step 2, an excess of Cbz-C12-NH₂ was added and full conversion was reached; the excess was afterwards removed by reacting with an isocyanate-resin and subsequent filtration. Reversed phase column purification was performed to purify the product resulting in a final yield of 63%. In step 3, the use of fresh triethylsilane resulted in swift deprotection with 97% yield. Step 4 was performed with a small excess of UPy-synthon, which was later removed via an amine resin and subsequent filtration, a yield of 72% was realised here. Without any further purification an overall yield of 40% was obtained. A similar synthesis route was conducted with a BOC-protected amine as tail group instead of a methoxy. Up until step 4 an overall yield of 64% was obtained. To yield the free amine a deprotection with hydrochloride/dioxane was performed on a fraction of the product, with this method 100% effective deprotection was obtained.

### Synthesis of Cationic Monofunctional Subunit C

An excess of TFA was added (approx. 15 ml) to 140 mg (141 μmole) pure BOC-protected UPy functionalized molecule. The reaction was performed for 1 hour while on ice and being stirred. The reaction was removed from ice and the mixture was dried with a nitrogen flow for 30 minutes. The dried mixture was dissolved in 10 ml of dH₂O and lyophilized for 72 hours, resulting in a pure, dry compound C with a yield of 70%.

### Synthesis of Neutral Monofunctional Subunit B

45 mg (45 μmole) of BOC-protected Monofunctional subunits was deprotected with TFA. After drying with a nitrogen flow the yellowish gel-like mixture was dissolved in 10 ml CHCl₃, after which an excess of DIPEA (approx. 3 ml) was added. Next, 14 μl (135 μmole, =3 eq.) of acetic anhydride was added and the reaction was stirred at RT overnight. After overnight reaction, the mixture was rotavapped. The dried compound was dissolved in approx. 3 ml dH₂O and 0.5 ml DMSO and dialysis was performed with a cellulose ester dialysis membrane with a molecular weight cutoff of 500-1000 Da. Dialysis was started in 1.5 L dH₂O while stirring gently. Over the course of 72 hours the dH₂O was refreshed three times. The compound was then lyophilized for 48 hours and dried under vacuum at 50° C. for 24 hours. This synthesis route resulted in a pure, dry compound B with a yield of 53% starting from the protected Monofunctional subunits.

### Synthesis of Dye Coupled Monofunctional Subunits

7.4 mg (7.5 μmole) of BOC-protected functionalized monofunctional subunit was deprotected with TFA. The resulting gel-like mixture was dissolved in 5 ml CHCl₃, after which an excess of DIPEA (approx. 2 ml) was added. To this mixture 5.47 mg (8.9 μmole, =1.2 eq.) of Cy5 Dye NHS ester (Lumiprobe, mw: 616.19 Da) was added. The reaction was protected from light using aluminum foil and stirred overnight. After overnight reaction the mixture was dried in the rotavap and dissolved in approx. 3 ml DMSO. Dialysis was performed using a regenerated cellulose membrane with a MWCO of 1 kDa. Dialysis was started in 1.5 liter dH₂O with 20% DMSO, after 18 hours the buffer was replaced with pure dH₂O and after 42 hours the water was refreshed once more. The compound was then lyophilized for 48 hours and this resulted in a dry, pure compound D with a yield of 39% starting from the protected UPy.

### Self-Assembly of Monofunctional Subunits

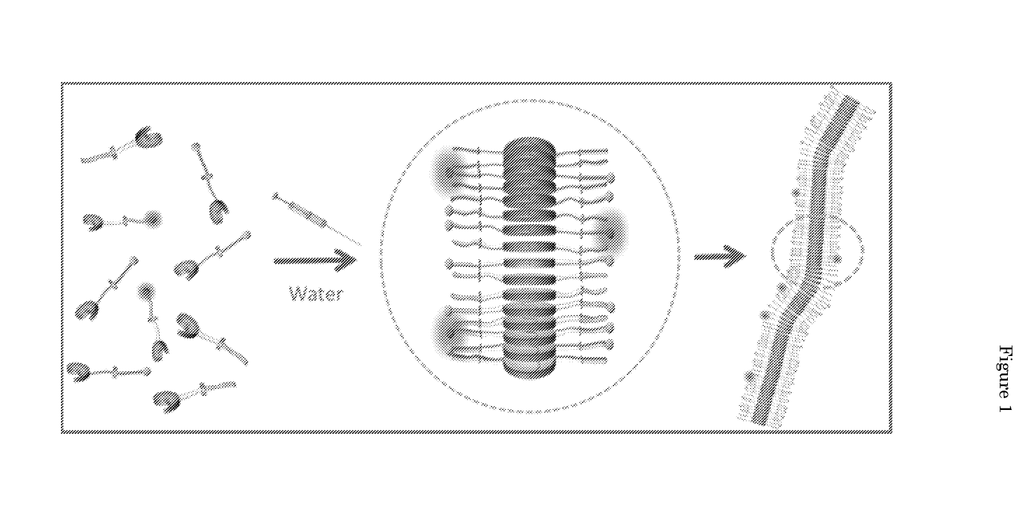

Self-assembly is triggered by injection into water, where a strong hydrophobic effect in combination with H-boding are the driving forces behind lateral stack formation (FIG. 1).

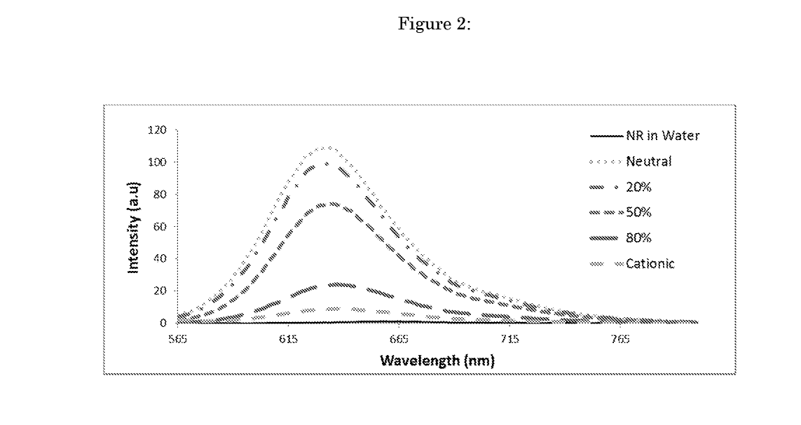

Fluorescent spectroscopy experiments using the fluorescent dye Nile Red (NR) were conducted. NR is a hydrophobic dye whose fluorescent characteristics depend on its environment. In water, as well as in most other polar solvents, NR is red shifted and barely fluorescent. Upon addition of NR in presence of assembled particles, NR quickly migrates to the hydrophobic interior of the supramolecular assembly and becomes fluorescently active. The fluorescence of NR is therefore a measure of the formation of hydrophobic pockets and thus of the formation of aggregated structures. This experiment is performed with varying ratios of cationic subunits. FIG. 2 shows the results of the NR encapsulation experiment.

FIG. 2 points out that NR is encapsulated by all the prepared monofunctional subunit assemblies, confirming availability of hydrophobic spaces and implying self-assembly. More intriguingly, a distinct trend can be observed in the fluorescent intensity in relation to the percentage of co-assembled cationic subunits. The fluorescence intensity is decreased by tenfold in the case of a full cationic assembly compared to the neutral assembly. An important observation is that the emission peak is at a constant wavelength. This means that the hydrophobic environment that NR is encapsulated in, is similar amongst all different assemblies; thus implying that the reason for the decrease in fluorescence is due to a decreased availability of hydrophobic spaces.

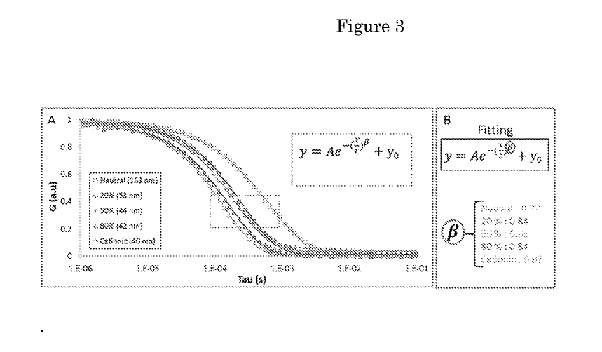

FIG. 3a shows the autocorrelation functions from DLS measurements and the stretched exponential fitted function from 1 angle (102°) and an estimation of the resulting size for a range of monofunctional subunit assemblies based on all the measured angles. In a unimodal sample with a normal distribution, the correlation follows a standard exponential decay. Here we use a stretched exponential decay function with the added parameter β. The stretched exponent β is introduced to take the possible polydispersity of the sample into account. In FIG. 3B the fitting function is displayed and the resulting parameter β for each sample. The value for β for neutral particle is 0.77, for 20% cationic particles is 0.84, for 50% cationic particles is 0.84 and for full cationic particles the value for β is 0.87. This value can be used as an indication of the dispersity of the sample.

A clear trend can be seen in the autocorrelation functions upon increasing the percentage of cationic subunits. The trend to the left indicates smaller particles. Size decrease upon introduction of repulsive electrostatic forces is hereby confirmed. Using a Matlab script—which runs a CONTIN analysis on the autocorrelation functions, subsequently calculates the diffusion coefficient and finally converts this value to size using the Stokes-Einstein equation—a size indication in the form of the hydrodynamic diameter is obtained. These values confirm the decrease in size. Introduction of a (small) percentage of cationic subunits has a great impact. Co-assembly of 20% cationic subunits is responsible for a decrease in mean size from 181 nm to 53 nm. Further increase of the percentage of cationic subunits results in even lower mean sizes. Interestingly, this data correlates with the data from the NR encapsulation experiment: smaller aggregates could be an explanation for the decreased availability of hydrophobic spaces upon co-assembly of cationic subunits.

Furthermore, a stretched exponential decay function was fitted through the autocorrelation functions. In a unimodal sample with a normal distribution a standard exponential decay function would fit perfectly. Here, we can judge the dispersity of the sample from the value of the stretched exponent β. From FIG. 3 we observe that the plotted stretched exponential functions fit the data sufficient. The β values from 0.84-0.87 indicate that the samples are unimodal but display a small increase in dispersity. A β value of 0.77 for the neutral stack indicates that this sample has a higher rate of dispersity.

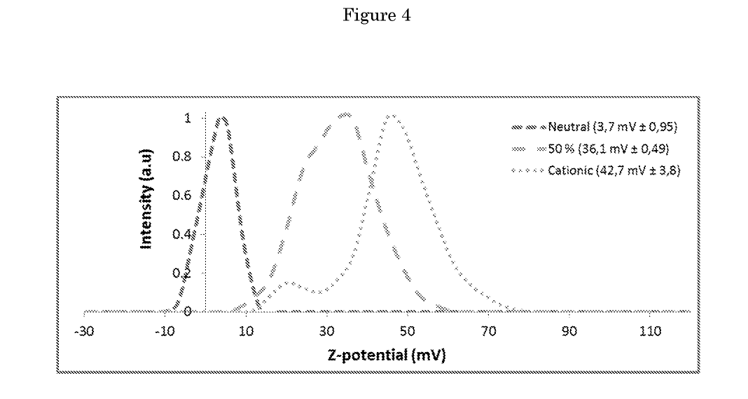

Z-potential of the assemblies were measured, as it is a property that directly correlates to the surface charge. Z-potentials were measured for neutral, 50% cationic and full cationic stacks and are shown in FIG. 4.

The assembly consisting of neutral subunits has a z-potential that is close to zero (3.7 mV) while both the 50% cationic (36.1 mV) and full cationic assemblies (42.7 mV) exhibit high positive z-potentials. The visible trend in z-potential is not linear with the percentage of cationic subunits as evidenced by the smaller increase in z-potential (6.6 mV increase) from 50% to full cationic compared to the 32.4 mV increase from neutral to 50% cationic. The experiment proves that co-assembly of cationic and negative subunits occurs. We succeeded in tuning the composition of the final assembly by varying the ratios of subunits.

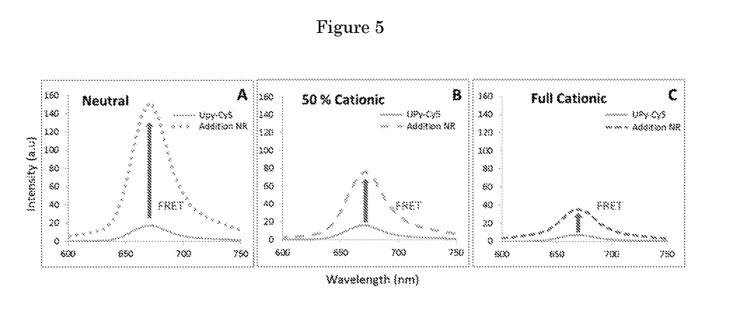

For a neutral, 50% cationic and full cationic stack FRET experiments were performed; the results are shown in FIG. 5.

All assemblies show an increased Cy5 emission intensity upon addition of NR, implying the incorporation of the Cy5 reporter subunit into the stacks. A FRET efficiency of 80% is determined. Occurrence of FRET effect upon encapsulation of NR and co-assembly of the Cy5 reporter monomer for neutral (A), 50% cationic (B) and full cationic (C) stacks. Excitation at 520 nm without NR present results only in very limited emission. Addition of NR greatly increases the fluorescence intensity, explained by the effective excitation of NR and the transferring of energy to Cy5 via FRET, resulting in increased Cy5 emission intensity.

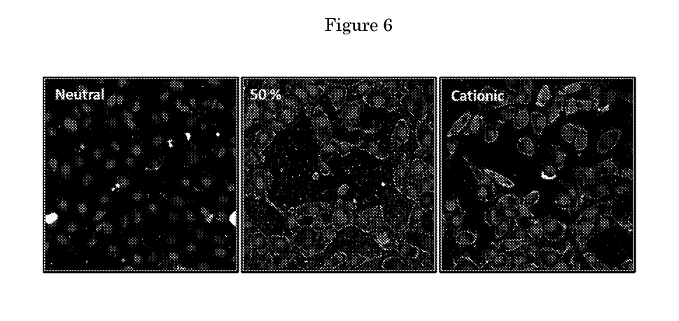

Before using the particles as delivery vehicles, their interaction with cultured cells was investigated. Careful investigation of the cellular binding and internalization using confocal microscopy can yield valuable information for further application and possible improvements with respect to siRNA delivery. Moreover, cytotoxicity is assessed, as it is a critical factor in deciding the future potential for application as siRNA delivery agent. For cellular internalization studies, multicomponent stacks consisting of either neutral, 50% cationic or only cationic subunits were prepared with 1% reporter subunit co-assembled. Cultured Human epithelial Kidney cells (HK2) were incubated with 500 μl medium containing a final subunit concentration of 10 μM. As an initial experiment, the location of particles was assessed after 10 minutes of incubation. In FIG. 6 we clearly see cell membrane binding for the 50% and full cationic particles and no binding in case of the neutral particles. It appears that electrostatic interactions seem to be the responsible forces behind cell membrane binding. Nuclei were stained with the dye Hoechst are depicted in blue while the particles are depicted in white, originating from the Cy5 dye reporter subunit.

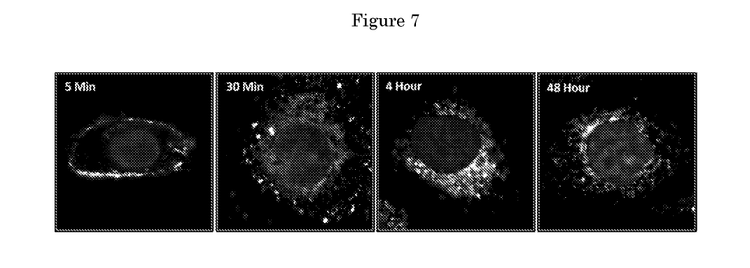

After 10 minutes of incubation and subsequent washing the neutral stacks have been completely washed away, apart from some large aggregates that stick either to some cells or the glass bottom. Both the 50% and full cationic samples the outer contours of the cell are visible due to the membrane binding of stacks. This observation implies that incorporation of 50% cationic subunits is sufficient to reach maximum cell membrane binding. It is clear that the cationic particles are internalized and furthermore we are able to obtain an idea of the fate of the particles over time. For this purpose, various images were acquired in a time lapse fashion from the moment of incubation to approximately 48 hours after incubation. In FIG. 7 four time points have been chosen that show a distinct localization and can therefore help clarify the internalization.

In the first few minutes after incubation with cationic particles they tend to bind to the cellular membrane. The particles seem to be distributed fairly even, coating the complete cell membrane. Over the course of the first thirty minutes the particles start to enter the cell and at time point 30 minutes it can be seen that part of the particles are still on the membrane while part is internalized and is, judging from the distinct morphology, possibly localized in the endoplasmic reticulum (ER). FIG. 6. Shows images of neutral, 50% cationic and full cationic particles after 10 minutes of incubation with HK2 cells. Nuclei are stained with Hoechst and shown in blue, particle are shown in white. Neutral particles show no cell binding while the 50% and full cationic particles are clearly bound to the cell membranes.

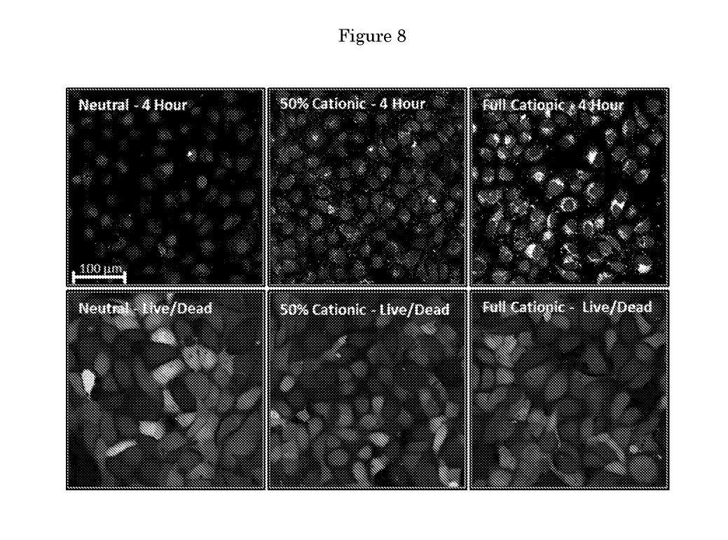

At hour 4 after incubation the particles are completely internalized and are mainly located at the perinuclear region. The distinct morphology of the ER is harder to recognize and at the same time the occurrence of small bright vesicles can be noticed. Images acquired 48 hours later indicate that the particles are still present in the cells, located as small vesicles freely located through the cell and in a small unidentified perinuclear region. FIG. 7 shows four images acquired at different time points during particle internalization. Four hours after incubation images were acquired of the neutral, 50% cationic and full cationic particle incubated samples for three reasons: 1) to confirm neutral particles are not internalized, 2) to estimate location and quantity of internalized cationic particles and 3) to assess the cytotoxicity using a live and dead staining. Detection of live cells was performed using calcein-AM: a cell-permeant compound that is converted in living cells to a green-fluorescent calcein. Staining of dead cells is conducted with the high affinity red nucleic acid stain ethidium bromide homodimer-1. Ethidium bromide homodimer-1 is not permeable to living cells. On the contrary, the membranes of dead cells can be penetrated due to an increased permeability where the fluorescent intensity of ethidium bromide increases 20-fold upon intercalating DNA. FIG. 8 shows the high amount of internalization of cationic particles after four hours incubation and a live/dead image from the exact same position at that time.

Similar as seen in the time-lapse image at four hours, the cationic particles are completely internalized without any membrane bound particle visible. Judging from the live/dead staining images the supramolecular particles did not induce cell death at the currently used concentration, meaning that conclusions on cytotoxicity are promising.

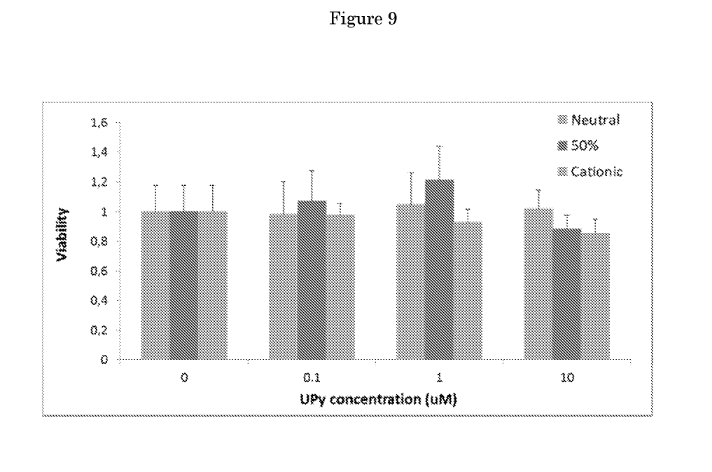

Cells were incubated with increasing concentrations of neutral, 50% cationic and full cationic particles. After 24 hours of incubation the tetrazole was added and reduced to the insoluble formazan by enzymes that are active in live cells. Subsequently 0.04 M HCl isopropanol was added to dissolve the insoluble purple formazan into a colored solution. The absorbance of this solution is measured at a wavelength of 590 nm and after normalization versus an untreated control sample, a quantification of the cell viability is obtained (FIG. 9).

The MTT assay data in FIG. 9 confirms the cytotoxicity observations. Slight variations exist between various samples and concentrations but none of the differences have been found significant.

During the investigation of the particles, information about the size, z-potential and composition were obtained. UPy concentration indicates subunit concentration. With the NR encapsulation experiment the formation of aggregates was confirmed and the z-potential data proved the aggregations were indeed mixtures of both neutral and cationic components. To some extent, both size and z-potential could be controlled by varying the ratio of these components. The information gathered during the NR encapsulation was furthermore used to design a FRET based experiment to proof the co-assembly of the Cy5 reporter subunit. Cell studies showed that both the 50% cationic and full cationic particles show a strong affection towards binding HK2 cells, while the neutral particles are refrained from binding. Over time, the cationic particles are completely internalized and accumulate in the perinuclear region. The promising cytotoxicity experiments during confocal imaging were confirmed by the quantitative MTT assay. As a result, the following important conclusions can be drawn:
Injection in water results in relatively quick, multicomponent aggregation.
Several properties are tunable by varying the ratio of individual components.
Cationic stacks exhibit a desired high positive z-potential.
Cy5 reporter monofunctional subunit is incorporated.
particles have the ability to encapsulate small hydrophobic molecules.
50% and full cationic particles show affection for binding the cellular membrane and are subsequently internalized. Treatment with various particles has no toxic effects on cells under the currently used conditions.

### Bifunctional Subunits as Crosslinking Hydrogelators

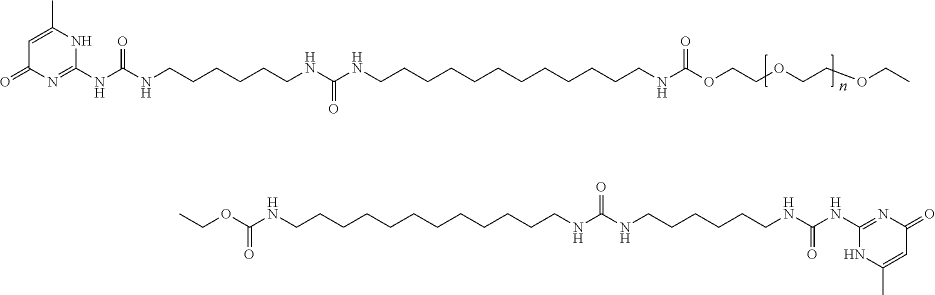

The bifunctional subunit (PEGdiUPy) hydrogelator was essentially made as described (Dankers et al. Adv. Mater. 2012, 24, 2703-2709)

### Materials & Methods

### NR Encapsulation

Nile red (NR) encapsulation measurements were performed on a Varian Cary Eclipse Fluorescence Spectrophotometer. 500 μl Mili-Q dH₂O samples with a final concentration of 50 μM subunits were prepared from cationic and neutral monofunctional subunit stock solutions in MeOH. Five variants were measured: neutral, 20% cationic, 50% cationic, 80% cationic and full cationic monofunctional subunits were injected in water and equilibrated for 2 hours by means of shaking. NR was added to the solution to a final concentration of 5 μM and samples were equilibrated by means of shaking for 5 minutes. The sample was then transferred to a cuvette, NR was excited at 550 nm with a laser power of 600 volt and the emission intensity was measured from 565 nm to 800 nm. 5 scans were performed of which the average was taken.

### Dynamic Light Scattering

Multi angle dynamic light scattering experiments were conducted on an ALV/CGS-3 MD-4 compact goniometer system equipped with a ALV-7004 real time correlator (solid state laser: λ=532 nm; 40 mW). Samples were prepared as following: Particle samples were equilibrated in 1 ml of filtered (0.2 μm filter) ultra-pure water in a final concentration of 50 μm. Experiments covered a range of angles between 36 and 148°. Each angle was measured in triplet, for 10 seconds, at 20° C., using a total of 4 detectors. Raw autocorrelation data of angle 102° was plotted to visualize the trend in decreasing correlation times. A stretched exponential function was fitted to this data to quantify the dispersity of the sample via the β value. To obtain an indication of the size the data was further analyzed using the available Matlab scripts, which make use of CONTIN analysis to calculate and plot the decay rate versus the square of wave vectors. The slope of this graph represents the diffusion coefficient and subsequently the hydrodynamic diameter is calculated via the Stokes-Einstein equation.

### Zeta-Potential

Zeta potential was measured on a Malvern Zetasizer Nano Z. Samples were prepared in a volume of 2 ml MiliQ ultrapure dH₂O. All samples were equilibrated prior to measurement by means of shaking for 2 hour. Approximately 1.5 ml of the samples was injected in a Malvern Disposable capillary cell (DTS1061). 70 runs of 10 seconds were performed at room temperature. Three consecutive measurements were performed and the mean and SD are shown. For the cationic, neutral and 50% cationic particles, samples with a concentration of 50 μM were prepared in a total volume of 2 ml. Therefore, 0.1 μmole of all three variants was added to dH₂O to a final volume of 2 ml from stock solutions in MeOH (stock cationic: 2 mM, stock neutral: 1 mM, stock 50%: 1.33 mM

### FRET Measurements

Samples of 500 μl Mili-Q dH₂O with 50 μM monofunctional subunits were prepared as described above. In addition, monofunctional subunit-Cy5 reporter was added to a final concentration of 0.5 μM (1%) prior to equilibration. The sample was excited at 520 nm with a laser power of 700 volt and emission intensity was measured from 540 nm to 800 nm. 5 Scans were performed of which the average was taken. Samples were measured both without and in the presence of Nile Red (final concentration 3 μM). Cy5-NHS ester and the monofunctional Cy5 subunit were both measured at a concentration of 0.5 μM as negative controls.

### Cell Culturing

HK-2 Cells were purchased from ATCC and cultured at 37° C. in 95% air/5% CO₂ atmosphere in Dulbecco's Modified Eagle Medium (DMEM) 41965-039 supplemented with 10% Foetal bovine serum (FBS) and 1% penicillin streptomycin (P/S). Cells were passed typically twice a week and for experiments cells ranging from passage 5 to 20 were used.

### Confocal Microscopy

Cell imaging was performed on a Leica TCS SP5X confocal microscope. For particle internalization studies cells were seeded 24 hours prior to imaging in a Lab-Tek Chambered #1.0 Borosilicate Coverglass System to a confluency of approximately 70%. The stacks were assembled in 100 μl Mili-Q dH₂O at a concentration of 50 μM containing 0.5 μM Cy5 reporter monomer (1%) and equilibrated for 2 hours by means of shaking. Right before imaging, the medium on the cells was discarded and cells were washed once with PBS. Afterwards, 400 μl of serum-free medium and subsequently the 100 μl sample was added, reaching a final subunit concentration of 10 μM. Sample and medium were gently mixed by pipetting up and down and afterwards imaging was started. Images were analyzed with ImageJ.

### MTT Assay

Thiazolyl Blue Tetrazolium Bromide (MTT) (Sigma-Aldrich #M2128) was used to perform toxicity assays. During the experiment a fresh working solution of 5 mg/ml MTT was prepared in PBS. This solution was filtered through a 0.2 μm filter and kept at 4° C. Cells were cultured under standard conditions and 15 k cells per well were seeded 24 hour prior to treatment in a BD Falcon 96-well Multiwell Plate. Samples were assembled in 25 μl Mili-Q dH₂O and equilibrated for 2 hours by means of shaking. Samples were then mixed with 100 μl DMEM medium containing 2% FBS and added to the cells. Final concentrations of 0, 0.1, 1 and 10 μM were tested, each in sevenfold. After the required incubation time, 13.75 μl (10%) of the MTT working solution was added to the wells (final volume 137.75 μl). Samples were incubated at 37° C. for approximately 2 hours and the medium was then removed. 150 μl of acidic isopropanol (isopropanol containing 0.04 M HCL) was added to the wells and mixed gently by pipetting up and down. The samples were then incubated at 37° C. for 20 minutes and gently mixed again afterwards. From each well, 100 μl was transferred to a Costar EIA/RIA 96 wells plate. The absorbance at 570 and 650 nm were measured with a Tecan Safire² microplate reader with 1000 reads at approximately 27° C. Background absorbance OD₆₅₀ was subtracted from OD₅₇₀, and the 7 samples per concentration were averaged. Values were normalized to the value for a concentration of 0.

Complex Formation Between Particles and siRNA

The complex formation between particles and siRNA to form supramolecular complexes is investigated. In order to achieve high delivery efficiency, complexes are preferred with a size below 100 nm and a positive net z-potential. With supramolecular particles, two distinct preparation techniques can be employed to obtain complexation. The first, named conventional method, is the addition of siRNA to pre-formed particles. Upon addition of siRNA, complexes form between cationic particle and negatively charged siRNA, resulting in larger electrostatic aggregates. Apart from the conventional method of complex preparation, supramolecular particles allow for a second preparation technique. Instead of adding siRNA to pre-formed supramolecular particles in water, injection of monofunctional subunits in water that already contains siRNA might result in different complexes. We propose that the negative charged siRNA can act as a ‘template’ for the monofunctional subunits to form particles polymerize on, possibly resulting in different aggregates. Both preparation strategies are tested on their ability to form supramolecular complexes with siRNA. Furthermore, multi angle DLS are conducted to analyze the resulting structures, and z-potential measurements to confirm whether the resulting supramolecular complexes display a net positive z-potential.

A generally accepted method to probe the formation of complexes with siRNA is via a gel retardation assay. Electrophoresis in agarose gel induces nucleic acids to migrate towards the anode. Upon strong complex formation with a cationic carrier, siRNA is firmly associated via electrostatic interactions with the cationic molecule. If these interactions are strong enough, siRNA withstands the electric field and is effectively retained by the carrier. Therefore, retainment of siRNA serves as evidence for complexation. A parameter that is used to describe complex formation is the ‘N/P’ ratio. N/P is the ratio of end amine groups in the particle (N) versus the total number of phosphates from the siRNA backbone (P). Common used N/P ratios for siRNA transfection with cationic particles lie between 2 and 20; lower ratios often result in partial or no complexation, while N/P ratios higher than 20 require large quantities of cationic material and often involve toxicity risks.

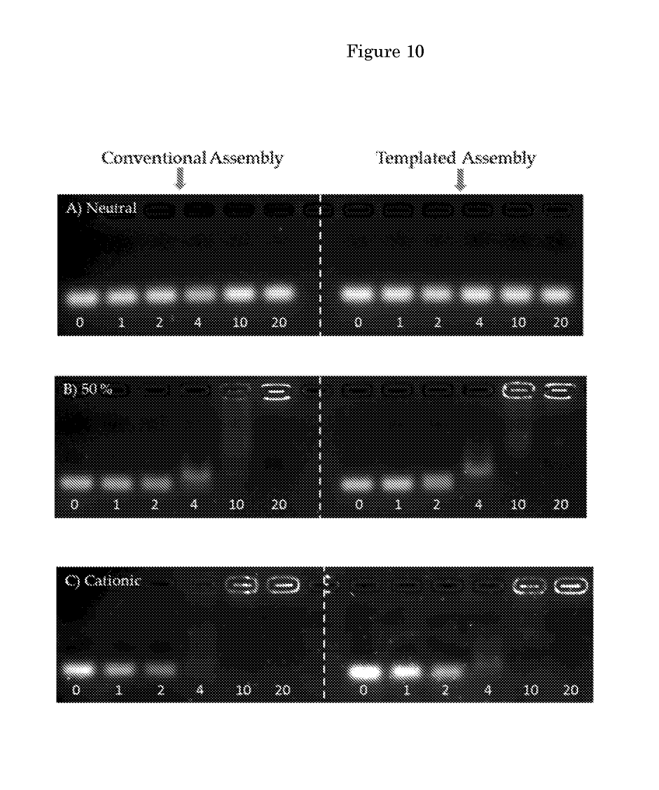

Agarose gel electrophoresis was performed on a neutral, 50% cationic and full cationic particles in complex with a fluorescently labeled siRNA at N/P ratios ranging from 1 to 20, prepared via the conventional and templated complexation methods. The resulting gels are demonstrated in FIG. 10; the left half of the gel presents complexes prepared via the conventional method while the right half are prepared via the templated assembly method. Values at the bottom of the gel indicate the N/P ratio used in the corresponding lane.

Judging from FIG. 10A, a completely neutral particle is not able to bind and retain siRNA when an electric field is applied, implying no complexation occurs independent of the amount of neutral monofunctional subunit used.

Looking at the 50% and cationic particles prepared using the conventional method there is a noticeable difference. A 50% cationic particle seems to start retaining siRNA at N/P=4 and keeps most, but not all, siRNA retained at N/P=10. The full cationic particle displays decent retardation at N/P=4 and complete retardation at N/P=10. Remarkably, using the templating assembly method siRNA is retained just as well for both the 50% and full cationic stacks. These results imply that pre-formation of particles is not a necessity in order to effectively bind siRNA. It learns that supramolecular particle formation occurs even in the presence of molecules with an opposite electrical charge.

Gel retardation assays showed that cationic polymers can effectively complex and retain siRNA, both via the conventional assembly method and the templating method. From multi angle dynamic light scattering it is known that the 50% cationic and full cationic polymers assemble to a size of approximately 50 nm.

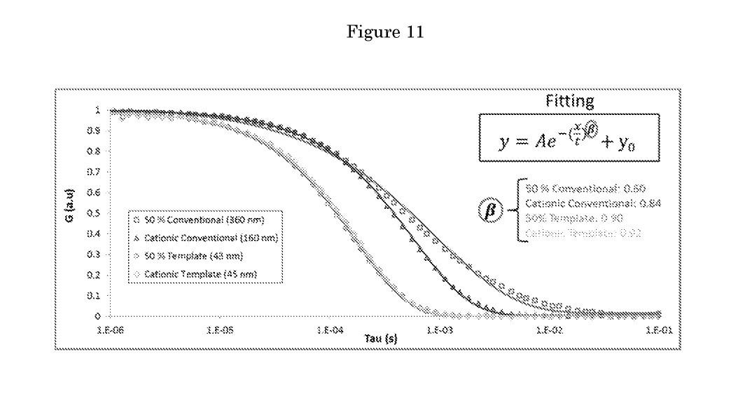

Using multi angle DLS, the resulting sizes of the supraplexes were determined and the two different assembly methods compared to learn the most suitable method for preparation of our desired supraplexes. Both the 50% and full cationic supraplexes were investigated; the neutral polymer is omitted since gel retardation proved it does not condensate siRNA. In FIG. 11 the autocorrelation data, the stretched exponential function fits, the stretched exponent parameter β and an estimation of the size of the supraplexes are shown. Autocorrelation functions from measurements at an angle of 102 degrees for 50% and full cationic supraplexes prepared via two preparation methods. Non-connecting markers represent the autocorrelation data and the solid line is the fitted stretched exponential. The values used for β to fit the stretched exponential function are displayed on the right.

Contrary to the gel retardation data, where the complex formation via the conventional method and the templated method yielded similar results, the DLS data shows a dramatic difference in properties of supraplexes between the two complexation methods. Both conventionally prepared particles are much larger in comparison to the template prepared particles. Via the templating method the resulting particle sizes, generated via CONTIN analysis, are similar to the sizes of the subunits before complexation. On the contrary: conventional assembly resulted in a fourfold size increase for the full cationic and tenfold size increase for the 50% cationic particles. Moreover, the β values close to 1 for the templated particles indicate a low rate of dispersity; even lower than the subunits exhibited prior to complexation. On the other hand, the cationic conventional particles gives a lower value for β, but is still accepted as a unimodal distribution. Especially the 50% cationic conventional assembly is a completely different sample. The quality of the fitted stretched exponential function is low and displays a low value for the stretched exponent β (0.60). This data suggests that this sample is actually bimodal. Based on these observations, it seems that complexation between pre-assembled supramolecular particles and siRNA results in large, polydisperse structures, implying that multiple particles condense multiple siRNA molecules into large, not well defined aggregates. On the other hand, supramolecular assembly in the presence of siRNA results in much smaller and better defined particles. For our goal of transfecting siRNA into human cells, the particles that result from the templated method are preferred.

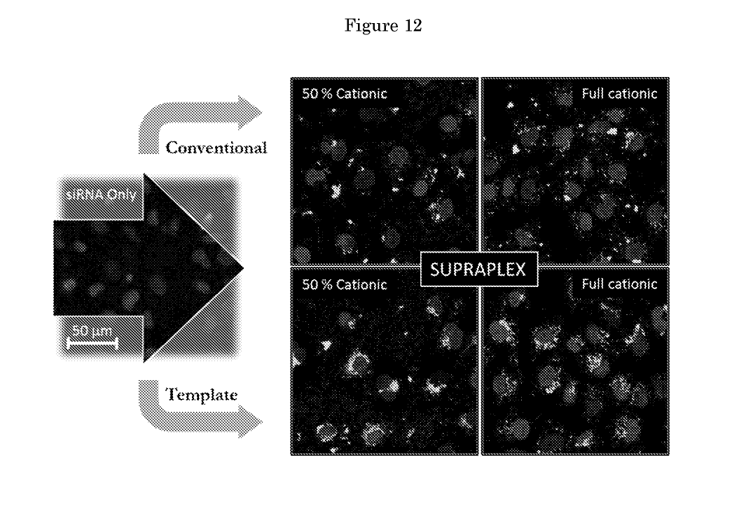

Important for the particles is that they display a cationic character: a necessity for binding with cellular membranes and to induce endocytosis. Both the 50% cationic and the full cationic particles exhibited promising z-potentials prior to complexation. The ideal magnitude of z-potential for a siRNA/particle delivery complex is not known. We measured z-potential for template prepared 50% and full cationic particles. The resulting 18.4±0.25 mV for the 50% cationic particle and 9.5±0.66 mV for the full cationic particle confirmed the cationic character of both particles. Also, these values are well in line with other cationic siRNA delivery systems. Next, fluorescently labeled siRNA was used to visualize whether the supraplex formation enables the delivery of siRNA into cells. Earlier it was demonstrated that sample preparation method has a great influence on the resulting supraplex properties. Here, samples were prepared via both preparation methods to find out if these difference in properties result in a difference in intracellular delivery as well. In FIG. 12 confocal microscopy images are displayed from HK2 cells treated for 1 hour with the four different samples. The siRNA is covalently bound to an Alexa488 dye and is shown in green.

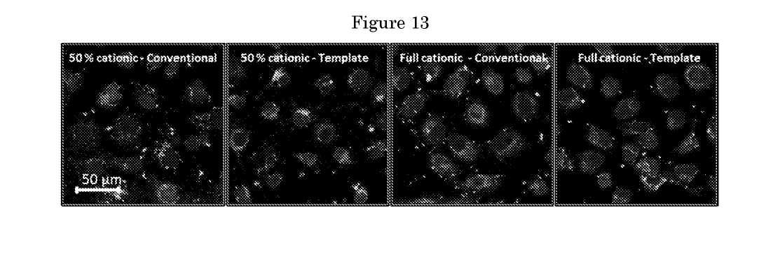

The naked, negatively charged siRNA has not been internalized by the cells. All tested particles have enabled transfer of siRNA into the cells, as evident from the green fluorescence inside the cells. At first sight, when comparing the different preparation methods and the different cationic supraplexes, no noticeable differences can be observed. Indeed, between 50% cationic and full cationic samples the results seem very similar. Yet, upon taking a closer look between the complexation methods, it is possible to perceive slight variations in the location of siRNA. In the case of the templated delivery, the siRNA seems to be more evenly distributed in small vesicles while in the conventional samples it looks like some aggregated structures which are not internalized are present. A second series of images was subsequently required after an increased incubation time and after washing the sample to get rid of aggregates and non-internalized siRNA, the resulting images are displayed in FIG. 13.

### Materials & Methods

### Gel Retardation Assay

All samples were mixed and equilibrated with 100 ng (6.4 pmol) fluorescently labeled (Alexa488) siRNA (Qiagen #1027284) at N/P ratios 0, 1, 2, 4, 10, 20 in a total volume of 30 μl (containing 5 μl 6× loading dye). particles were prepared by addition and equilibration of siRNA in water and subsequent addition and equilibration of subunits (template method) or vice versa (conventional method). For the neutral monofunctional subunits amounts were used similar to if it was a full cationic subunit, for one cannot calculate with N/P ratios for the neutral subunit. Samples were run on a 1.5% agarose gel at 70 volt for 30 minutes. The gel was then imaged using an ImageQuant 350 Gel imaging system using the Sybrsafe filter.

### RNA Interference

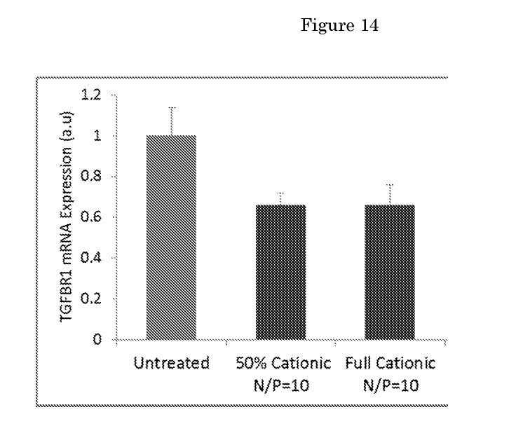

In our experiments, we employ RNA interference to alter the gene expression at the mRNA level. Subsequent quantification of the changes in mRNA levels yields the silencing efficiency. It was chosen to target the mRNA coding for the transforming growth factor beta receptor 1 (TGFBR1). Tested are the 50% cationic stack at N/P=10 and full cationic stack at N/P=10. A silencing experiment was performed with supraplexes prepared via the conventional method with siRNA versus TGFBR1, the results are illustrated in FIG. 14. Results of silencing the TGFBR1. 50% Cationic particles, full cationic particles were prepared via the conventional preparation method at an N/P ratio of 10. Samples are normalized versus untreated cells and represent mean±SD, n=3.

### RNA Extraction and DNA Synthesis

RNA extraction was performed using a High Pure RNA isolation Kit (Roche, 828 665 001) following manufacturers protocol. Reverse transcription was performed using an iScript cDNA synthesis kit (Biorad, #170-8891) following manufacturers protocol. qPCR was performed on a Biorad MyiQ with iQ5 software using a Sybr Green mastermix as detection agent. All mRNA expression values are normalized against the household gene GAPDH. Dixon's Qtest (90%) was applied to identify outliers.

### Silencing of TGFBR1

HK2 cells were seeded in a 24 wells plate in 1 ml supplemented medium so that the next day a confluency of 50-70% was reached. The next day, subunits+transfection reagent+anti TGFBR1 siRNA (610 ng siRNA=0.045 nmol) complexes were prepared via the chosen preparation method at the desired N/P ratio in 200 μl dH₂O. After shaking for 3 hours, the 200 μl samples were mixed with 800 μl medium (supplemented with 2% FBS without penicillin streptomycin (P/S)) so that a final concentration of 45 nM siRNA was reached. Cells were washed with PBS once and subsequently 1 ml of the sample-medium mixture was added. Approximately 4 hours after transfection, medium was discarded and fresh medium containing 10% FBS and 1% P/S was added. Approximately 48 hours after transfection the RNA was extracted and immediately afterwards transcribed to cDNA. RNA was stored at −80° C. and cDNA was stored at −30° C. Quantitative polymerase chain reaction was performed to quantify TGFBR1 mRNA expression levels. FIG. 14 shows the results of the silencing the TGFBR1. 50% Cationic particles, full cationic particles were prepared via the conventional preparation method at an N/P ratio of 10. Samples are normalized versus untreated cells and represent mean±SD, n=3. As can be seen 50% and 100% cationic particles are able to introduce siRNA into the cell and let them silence TGFBR1 expression.

### REFERENCES

1. Malvern-Instruments, “Dynamic Light Scattering: An Introdcution in 30 Minutes”, Malvern-Instruments, http://www.malvern.com/common/downloads/campaign/MRK656-01.pdf.
2. Malvern-Instruments, “Zeta Potential An Introduction in 30 Minutes”, Malvern Instruments, http://www.nbtc.cornell.edu/facilities/downloads/Zeta%20potential%20-%20An%20introduction%20in%2030%20minutes.pdf.
3. Z.-M. Inc, “Zeta Potential: A Complete Course in 5 Minutes”, Zeta-Meter Inc., http://www.zeta-meter.com/5 min.pdf.
4. P. Held, “An Introduction to Fluorescence Resonance Energy Transfer (FRET) Technology and its Application in Bioscience”, BioTek Instruments, (20 Jun. 2005). http://www.biotek.com/assets/tech_resources/FRET%20Application%20Guide.pdf.
5. L. Albertazzi, M. Serresi, A. Albanese, F. Beltram, Dendrimer internalization and intracellular trafficking in living cells. Molecular pharmaceutics 7, 680 (Jun. 7, 2010).

1. Particle comprising a supramolecular complex comprising a monofunctional subunit with formula (I)
4H-L₁-F₁-L₂-F₂—P-E-Z
wherein
4H is a quadruple hydrogen bonding unit,
L₁ and L₂ are selected from the group consisting of C₁₋₅₀ alkyl and C₂₋₅₀ alkenyl;
F₁ is —NH—C(═O)—NH—;
F₂ is selected from the group consisting of —NRₐ—C(X)—NRₐ— and —NRₐ—C(X)—X—;
X is O or S;
Rₐ is hydrogen, or C₁₋₁₂ alkyl;
P is a polyethyleneglycol linker with 0 to 1000 ethyleneglycol monomers;
E is a direct bond, linker L_{E}, linker P_{E}, or combinations of L_{E} and P_{E} linkers;
L_{E} is a linker as defined for L₁ or L₂;
P_{E} is a polyethyleneglycol linker as defined with for polyethyleneglycol linker P;
Z is a functional group selected from the group comprising a neutral moiety, ionic moiety, peptide, therapeutic moiety, imaging agent, fluorescent moiety, targeting moiety, endosomal escape agent moiety, cell-penetrating peptides, antigen, adjuvant, antibody, wherein at least 1% of the monofunctional subunits comprise a cationic Z moiety.

2. Particle according to claim 1 wherein at least 10 subunits of formula (I) are present.

3. Particle according to any claim 1 wherein at least 10% of the monofunctional subunits are cationic.

4. Particle according to claim 1 wherein the z potential of the particle is between 0 and +50 v.

5. Particle according to claim 1 wherein the hydrodynamic diameter of the particle is between 0.2 and 1000 nm.

6. Particle according claim 1 wherein the monofunctional subunit has formula (III) 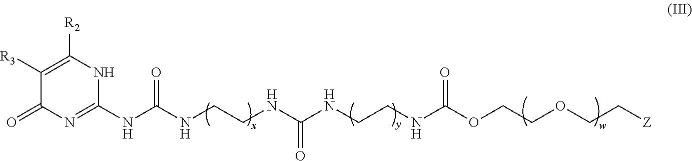
x is an integer from 1 to 50,
y is an integer from 1 to 50
w is an integer from 0 to 1000,
R₂ and R₃ are each independently a hydrogen, C₁₋₂₄alkyl, C₂₋₂₄alkenyl, C₂₋₂₄ alkynyl, or C₃₋₁₂-cycloalkyl.

7. Particle according to claim 1 further comprising a bifunctional subunit with formula (II):
4H-L₁-F₁-L₂-F₂-G-F₂-L₂-F₁-L₁-4H (II)
wherein G is a polyethyleneglycol linker with a molecular weight of at least 500 Dalton and wherein L₁, F₁, L₂, and F₂ are as defined in claim 1.

8. Particle according to claim 7 wherein the bifunctional subunit with formula (II) is present in at least 2 wt %.

9. Particle according to claim 7 in the form of a hydrogel.

10. Process for making a particle according to claim 1 comprising the step:
i) adding said subunit to water.

11. Method for entering or labelling a cell comprising contacting a particle according to claim 1 with a cell.

12. Method of delivering a drug to a cell comprising contacting a drug bound to a particle according to claim 1 with a cell.

13. Method of administering an imaging agent comprising administering an imaging agent bound to a particle according to claim 1 to a subject.

14. Method of administering a hydrogel prolong release system of a drug comprising administering to a subject a drug bound to a particle according to claim 1, whereby the drug is released over a prolonged period of time.

15. Method of treating a damaged tissue comprising implanting a plurality of particles according to claim 1 into a damaged issue, whereby the particles form a hydrogel that provides mechanical support for the damaged tissue.

16. Particle according to claim 2 wherein at least 20 subunits of formula (I) are present.

17. Particle according to claim 2 wherein at least 50 subunits of formula (I) are present.

18. Particle according to claim 3 wherein the particle comprises between 20% and 80% cationic subunits.
